SEQUENCE LISTING (10 sequences)

SEQ ID NO: 1 - DNA, 2269 bp, Homo sapiens
  misc_feature (61)..(61): “n” is A,T,C or G
atgtctcgctccgtggccttagctgtgctcgcgctactctctctttctggcctggaggccnggtggaggtggttctggaggaggcggttcgggcggaggtggtagtatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtattatactgaattcacccccactgaaaaagatgagtatgcctgccgtgtgaaccacgtgactttgtcacagcccaagatagttaagtgggatcgagacatgggaggcggaggatctggtggtggaggttctggtggtgggggatctggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggcgcctacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgggtggaggcggatctggcggcggaggatctggaggaggtggatctgggggcggtggtagtggcctgaatgacatctttgaagcccagaaaatcgaatggcacgaaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctccaggacggcgagttcatctacaaggtgaagctgcgcggcaccaacttcccctccgacggccccgtaatgcagaagaagacaatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggtggaggtggttctggaggaggcggttcgagcagccagccgaccattccgattgtgggcattattgcgggcctggtgctgtttggcgcggtgattaccggcgcggtggtggcggcggtgatgtggcgtcgtaaaagcagcgatcgtaaagattataaagatgatgatgataaataatag

SEQ ID NO: 2 - protein, 755 aa, Homo sapiens
  MISC_FEATURE (21)..(21): “Xaa” is any Amino Acid.
MSRSVALAVLALLSLSGLEAXNGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPGGGGSGGGGSGGGGSGGGGSGLNDIFEAQKIEWHEMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGGGGSGGGGSSSQPTIPIVGIIAGLVLFGAVITGAVVAAVMWRRKSSDRKDYKDDDK

SEQ ID NO: 3 - protein, 20 aa, Homo sapiens
MSRSVALAVLALLSLSGLEA

SEQ ID NO: 4 - protein, 99 aa, Homo sapiens [sequence omitted]

SEQ ID NO: 5 - protein, 276 aa, Homo sapiens
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEP

SEQ ID NO: 6 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 8 aa, Artificial Sequence
  Synthetic Sequence
NRVRQGYS

SEQ ID NO: 8 - protein, 5 aa, Artificial Sequence
  Synthetic Sequence
GGGGS

SEQ ID NO: 9 - protein, 9 aa, Artificial Sequence
  Synthetic Sequence
NLVPMVATV

SEQ ID NO: 10 - protein, 9 aa, Artificial Sequence
  Synthetic Sequence
GILGFVFTL

(19) United States
(12) Patent Application Publication
(10) Pub. No.: US 2023/0052675 A1
(43) Pub. Date: Feb. 16, 2023

(54) CELLULAR PLATFORM FOR RAPID AND COMPREHENSIVE T-CELL IMMUNOMONITORING

(71) Applicant: Albert Einstein College of Medicine, Bronx, NY (US)

(72) Inventors: Ronald D. Seidel, III, Bronx, NY (US); Rodolfo J. Chaparro, Bronx, NY (US); Brandan S. Hillerich, Ithaca, NY (US); Steven C. Almo, Pelham, NY (US)

(73) Assignee: Albert Einstein College of Medicine, Bronx, NY (US)

(21) Appl. No.: 17/827,024

(22) Filed: May 27, 2022

Related U.S. Application Data
(63) Continuation of application No. 15/110,384, filed on Jul. 7, 2016, now abandoned.
(60) Provisional application No. 61/929,651, filed on Jan. 21, 2014.

(51) Int. Cl. G01N 33/569; C07K 14/74; C12Q 1/6881

(52) U.S. Cl. CPC G01N 33/56972; C07K 14/70539; C12Q 1/6881; C07K 2319/03; C07K 2319/30; G01N 2333/15; G01N 2333/155

(57) ABSTRACT

Methods and systems for the efficient and systematic identification of the repertoire of T-cell epitopes.

### CROSS-REFERENCE TO RELATED APPLICATIONS

This application is a continuation of Ser. No. 15/110,384, filed Jul. 7, 2016, which is a national stage filing under 35 U.S.C. § 371 of International Patent Application No. PCT/US2015/012160, filed Jan. 21, 2015, which claims the benefit of U.S. Provisional Application No. 61/929,651, filed Jan. 21, 2014, which applications are incorporated by reference herein in their entirety.

### STATEMENT OF GOVERNMENT SUPPORT

This invention was made with government support under grant numbers 3U54GM094662-02 and 5U01GM094665-02 awarded by NIGMS, National Institutes of Health. The government has certain rights in the invention.

### BACKGROUND OF THE INVENTION

Throughout this application various publications are referred to in square brackets. Full citations for these references may be found at the end of the specification. The disclosures of these publications, and all patents, patent application publications and books referred to herein, are hereby incorporated by reference in their entirety into the subject application to more fully describe the art to which the subject invention pertains.

Remarkable growth has been made over the past decade in the development and application of genomic [13-15] and proteomic technologies [16-19] for the identification of molecular signatures associated with clinically important disease states and differential responses to therapies. These advances hold the promise of personalized diagnostics [20]. As an example, Adaptive Biotechnologies utilizes high throughput sequencing of the T cell receptor (TCR) beta chain hypervariable region to provide researchers with a full analysis of the TCR repertoire within a sample [21]. This venture capital-funded effort is presently a fee-for-service enterprise, with a projected market depth for biomarker discovery of $300 million. The rapid progress in high throughput technologies has been paralleled by the stepwise clinical development of biologics (e.g., monoclonal antibodies, therapeutic proteins, and peptides) [22-25], and has revolutionized the treatment of immune borne diseases. For example, unlike traditional vaccines, which boost immunity primarily via antibody responses, Genocea Biosciences is developing novel, biologics-based vaccines focused on generating robust T-cell responses against intracellular pathogens. Likewise, Apitope, a European biotechnology company, is developing therapeutic peptides for the treatment of autoimmune diseases in which T-cells play a key pathogenic role. Apitope recently partnered with Merck-Serono for the continued development of their flagship MS peptide therapeutic. Efforts seeking to monitor, enhance or alter T-cell immunity will depend heavily on the ability to identify clinically relevant T-cell epitopes.

At the core of the molecular events comprising a CD8-mediated adaptive immune response is the engagement of the T-cell receptor (TCR) with a small peptide antigen non-covalently presented by a major histocompatibility complex (MHC) molecule, referred to as a T-cell epitope. This represents the immune system's targeting mechanism and is a requisite molecular interaction for T-cell activation and effector function. During T-cell development, a genomic editing process results in the expression of a unique TCR on every immune cell, with an estimated depth of over 3 million unique sequences [1] and accounts for the enormous diversity of antigens to which T-cells can respond. However, T-cell epitopes have historically been difficult to study, as each TCR requires individual characterization with respect to specificity as well as the development of custom reagents (e.g., tetramers) for further study. Clinically, this challenge is compounded by the fact that immune responses typically involve many T-cell specificities, for example targeting multiple viral antigens to effect viral clearance for a single pathogen response. Thus, the ability to systematically identify the entire ensemble of epitopes for a given disease state represents a unique opportunity for the development of diagnostics and potential highly targeted therapeutics against infectious diseases, autoimmunity and cancers.

There exists a number of experimental approaches for epitope discovery, which include the screening of expression [3, 4] and synthetic peptide libraries [5, 6], positional scanning libraries [7], pMHC microarrays [8], as well as mass spectrometric identification of naturally-occurring epitopes [9-11]. Marrack and Kappler developed a baculovirus-infected insect cell strategy as a display platform for class I MHC molecules covalently bound to a library of potential peptide mimotopes [4]. Mimotopes differ in sequence from the unknown peptide epitope, but they are nevertheless recognized by the specific CD8 T-cell receptor. However, it is often challenging to link the identified mimotope to the natural epitope. Moreover, the baculoviral display system requires 5-10 time-consuming rounds of cell sorting, viral generation, expansion and reinfection to resolve a mimotope, coupled with a requirement to purify and tetramerize the cognate TCR. Partially addressing these issues, Newell et al leveraged heavy-isotope tagging of traditional MHC tetramers combined with flow cytometry and mass spectroscopy (termed mass cytometry) to screen a small set of pMHC tetramer combinations directly from a human blood sample with astonishing sensitivity, although this technology is presently limited to ˜100 such combinations per assay [12]. Each of these approaches has contributed valuable insights into T-cell epitopes; however, these methods are slow, labor-intensive and require a high degree of user skill.

The present invention addresses this need for new and improved technologies for the efficient and systematic identification of the repertoire of T-cell epitopes.

### SUMMARY OF THE INVENTION

This invention provides an isolated suspension-adapted cell transduced by or transfected with a heterologous nucleic acid comprising, in 5′ to 3′ order:

a leader oligonucleotide sequence, contiguous with
an oligonucleotide sequence encoding an 8, 9, 10, 11 or 12 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain sequence, contiguous with
an oligonucleotide sequence encoding a third linker, contiguous with
an oligonucleotide sequence encoding a fluorescent protein, contiguous with
an oligonucleotide sequence encoding a fourth linker, contiguous with
an oligonucleotide sequence encoding a Major Histocompatibility Complex heavy chain transmembrane domain.

This invention also provides isolated suspension-adapted cell expressing an expression product of a heterologous nucleic acid transduced or transfected therein, which expression product comprises, in N-terminal to C-terminal order:

an 8, 9, 10, 11 or 12 amino acid peptide, contiguous with
a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a fluorescent protein, contiguous with
a fourth linker peptide sequence, contiguous with
a Major Histocompatibility Complex heavy chain transmembrane domain.

Also provided is a recombinant nucleic acid comprising, in 5′ to 3′ order:

a sequence encoding a leader oligonucleotide sequence, contiguous with
an oligonucleotide sequence encoding an 8, 9, 10, 11 or 12 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
a Major Histocompatibility Complex heavy chain sequence, contiguous with
a third linker peptide sequence, contiguous with
a fluorescent protein, contiguous with
a fourth linker peptide sequence, contiguous with
a Major Histocompatibility Complex heavy chain transmembrane domain.

Also provided is a method of identifying a T-cell epitope comprising: contacting a T-cell with a plurality of isolated suspension-adapted cells comprising at least two cells, each expressing an expression product of a heterologous nucleic acid transduced or transfected therein, each of which expression products comprises an 8, 9, 10, 11 or 12 amino acid peptide, contiguous with first linker peptide sequence, contiguous with a beta 2 microglobulin sequence, contiguous with a second linker peptide sequence, contiguous with a Major Histocompatibility Complex heavy chain sequence, contiguous with a third linker peptide sequence, contiguous with a fluorescent protein, contiguous with a fourth linker peptide sequence, contiguous with a Major Histocompatibility Complex heavy chain transmembrane domain, wherein the plurality of isolated suspension-adapted cells expresses at least two different encoded 8, 9, 10, 11 or 12 amino acid peptides among the cells thereof under conditions permitting T-cells to conjugate with the 8, 9, 10, 11 or 12 amino acid peptides;

recovering T-cell(s) which have formed a conjugate with a suspension-adapted cell;
recovering DNA from the recovered T-cell(s);
sequencing the recovered DNA;
identifying the 8, 9, 10, 11 or 12 amino acid peptide(s) encoded for in the DNA, so as to thereby identify a T-cell epitope.

Also provided is an isolated suspension-adapted cell transduced by or transfected with a heterologous nucleic acid comprising, in 5′ to 3′ order:

a leader oligonucleotide sequence, contiguous with
an oligonucleotide sequence encoding an 8, 9, 10, 11 or 12 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain
sequence, contiguous with
an oligonucleotide sequence encoding a third linker, contiguous with
an oligonucleotide sequence encoding a Major Histocompatibility Complex heavy chain transmembrane domain.

Also provided is an isolated suspension-adapted cell expressing an expression product of a heterologous nucleic acid transduced or transfected therein, which expression product comprises, in N-terminal to C-terminal order:

an 8, 9, 10, 11 or 12 amino acid peptide, contiguous with
a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a Major Histocompatibility Complex heavy chain transmembrane domain.

A recombinant nucleic acid is provided comprising, in 5′ to 3′ order:

a sequence encoding a leader oligonucleotide sequence, contiguous with
an oligonucleotide sequence encoding an 8, 9, 10, 11 or 12 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
a Major Histocompatibility Complex heavy chain sequence, contiguous with
a third linker peptide sequence, contiguous with
a Major Histocompatibility Complex heavy chain transmembrane domain.

Also provided is a method of identifying a T-cell epitope comprising contacting a T-cell with a plurality of isolated suspension-adapted cells comprising at least two cells, each expressing an expression product of a heterologous nucleic acid transduced or transfected therein, each of which expression products comprises an 8, 9, 10, 11 or 12 amino acid peptide, contiguous with first linker peptide sequence, contiguous with a beta 2 microglobulin sequence, contiguous with a second linker peptide sequence, contiguous with a Major Histocompatibility Complex heavy chain sequence, contiguous with a third linker peptide sequence, contiguous with a Major Histocompatibility Complex heavy chain transmembrane domain, wherein the plurality of isolated suspension-adapted cells expresses at least two different encoded 8, 9, 10, 11 or 12 amino acid peptides among the cells thereof under conditions permitting T-cells to conjugate with the 8, 9, 10, 11 or 12 amino acid peptides;

recovering T-cell(s) which have formed a conjugate with a suspension-adapted cell;
recovering DNA from the recovered T-cell(s);
sequencing the recovered DNA;
identifying the 8, 9, 10, 11 or 12 amino acid peptide(s) encoded for in the DNA,
so as to thereby identify a T-cell epitope.

Also provided is an isolated suspension-adapted cell transduced by or transfected with a heterologous nucleic acid comprising, in 5′ to 3′ order:

a leader oligonucleotide sequence, contiguous with
an oligonucleotide sequence encoding a 5 to 20 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain sequence, contiguous with
an oligonucleotide sequence encoding a third linker, contiguous with
an oligonucleotide sequence encoding a fluorescent protein or encoding an immunoglobulin Fc domain, contiguous with
an oligonucleotide sequence encoding a fourth linker, contiguous with
an oligonucleotide sequence encoding a mammalian transmembrane domain.

Also provided is an isolated suspension-adapted cell expressing an expression product of a heterologous nucleic acid transduced or transfected therein, or a membrane-bound portion of such cell expressing the expression product, which expression product comprises, in N-terminal to C-terminal order:

a 5 to 20 amino acid peptide, contiguous with
a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a fluorescent protein or a sequence of an immunoglobulin Fc domain, contiguous with
a fourth linker peptide sequence, contiguous with
a mammalian transmembrane domain.

Also provided is an isolated suspension-adapted cell expressing an expression product of a heterologous nucleic acid transduced or transfected therein, or a membrane-bound portion of such cell expressing the expression product, which expression product comprises, in N-terminal to C-terminal order:

a 5 to 20 amino acid peptide, contiguous with
a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a fluorescent protein or a sequence of an immunoglobulin Fc domain, contiguous with
a fourth linker peptide sequence, contiguous with
a mammalian transmembrane domain.

A plurality of the isolated suspension-adapted cells or a plurality of membrane-bound portions of such cells expressing the expression product, wherein the plurality comprises at least two different encoded 5 to 20 amino acid peptides, is also provided.

A (i) virus-like particle or (ii) virus, produced by an isolated suspension-adapted cell as described herein is provided, which virus like particle or a virus is physically associated via a cell membrane portion having attached thereto, by a mammalian transmembrane domain, an expression product comprising in N-terminal to C-terminal order:

a 5 to 20 amino acid peptide, contiguous with
a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a fluorescent protein or a sequence of an immunoglobulin Fc domain, contiguous with
a fourth linker peptide sequence, contiguous with
the mammalian transmembrane domain, contiguous with
a viral packaging sequence.

A plurality of the virus-like particles described, or of the viruses described, is also provided.

Also provided is a recombinant nucleic acid comprising, in 5′ to 3′ order:

a sequence encoding a leader oligonucleotide sequence, contiguous with
an oligonucleotide sequence encoding a 5 to 20 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain sequence, contiguous with
an oligonucleotide sequence encoding a third linker peptide sequence, contiguous with
an oligonucleotide sequence encoding a fluorescent protein or an immunoglobulin Fc domain, contiguous with
an oligonucleotide sequence encoding a fourth linker peptide sequence, contiguous with
an oligonucleotide sequence encoding a mammalian transmembrane domain.

Also provided is an isolated suspension-adapted cell transduced by or transfected with a heterologous nucleic acid comprising, in 5′ to 3′ order:

an oligonucleotide sequence encoding a first B2M leader sequence,
contiguous with an oligonucleotide sequence encoding a preselected 5 to 20 amino acid peptide, contiguous with an oligonucleotide sequence encoding a first amino acid linker sequence, contiguous with an oligonucleotide sequence encoding a sequence of amino acids identical to a human native B2M peptide sequence,
contiguous with an oligonucleotide sequence encoding a second amino acid linker sequence,
contiguous with an oligonucleotide sequence encoding a preselected second peptide sequence,
contiguous with an oligonucleotide sequence encoding a third amino acid linker,
contiguous with an oligonucleotide sequence encoding a second B2M leader sequence,
contiguous with an oligonucleotide sequence encoding a sequence of amino acids identical to a MHC heavy chain,
contiguous with an oligonucleotide sequence encoding a fourth amino acid linker,
contiguous with an oligonucleotide sequence encoding a sequence of amino acids identical to an immunoglobulin Fc domain,
contiguous with an oligonucleotide sequence encoding a fifth linker,
contiguous with an oligonucleotide sequence encoding a mammalian transmembrane domain.

Also provided is an isolated suspension-adapted cell expressing an expression product of a heterologous nucleic acid transduced or transfected therein, or a membrane-bound portion of such cell expressing the expression product, which expression product comprises a recombinant polypeptide construct comprising (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence, wherein (i) is bound by one, or more than one, disulfide bond to (ii) a sequence of amino acids having the sequence of a MHC heavy chain contiguous with a fourth amino acid linker sequence contiguous with a sequence of amino acids identical to an immunoglobulin Fc domain contiguous with a fifth amino acid linker, contiguous with a mammalian transmembrane domain.

Also provided is an isolated suspension-adapted cell transduced by or transfected with a virus, plasmid or viral vector comprising a heterologous nucleic acid comprising, in 5′ to 3′ order:

an oligonucleotide sequence encoding a first B2M leader sequence,
contiguous with an oligonucleotide sequence encoding a preselected 5 to 20 amino acid peptide, contiguous with an oligonucleotide sequence encoding a first amino acid linker sequence, contiguous with an oligonucleotide sequence encoding a sequence of amino acids identical to a human native B2M peptide sequence,
contiguous with an oligonucleotide sequence encoding a second amino acid linker sequence,
contiguous with an oligonucleotide sequence encoding a preselected second peptide sequence,
contiguous with an oligonucleotide sequence encoding a third amino acid linker,
contiguous with an oligonucleotide sequence encoding a second B2M leader sequence,
contiguous with an oligonucleotide sequence encoding a sequence of amino acids identical to a MHC heavy chain,
contiguous with an oligonucleotide sequence encoding a fourth amino acid linker,
contiguous with an oligonucleotide sequence encoding a sequence of amino acids identical to an immunoglobulin Fc domain,
contiguous with an oligonucleotide sequence encoding a fifth linker,
contiguous with an oligonucleotide sequence encoding a mammalian transmembrane domain, contiguous with an oligonucleotide encoding a viral packaging sequence.

A (i) virus like particle or (ii) virus, produced by the cell of claim 46, which virus like particle or a virus is physically associated via a cell membrane portion having attached thereto, by a mammalian transmembrane domain, an expression product comprising in N-terminal to C-terminal order:

a recombinant polypeptide construct comprising (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence, wherein (i) is bound by one, or more than one, disulfide bond to (ii) a sequence of amino acids having the sequence of a MHC heavy chain contiguous with a fourth amino acid linker sequence contiguous with a sequence of amino acids identical to an immunoglobulin Fc domain contiguous with a fifth amino acid linker, contiguous with a mammalian transmembrane domain, contiguous with a viral packaging sequence.

Also provided is a recombinant nucleic acid comprising, in 5′ to 3′ order:

an oligonucleotide sequence encoding a first B2M leader sequence,
contiguous with an oligonucleotide sequence encoding a preselected 5 to 20 amino acid peptide, contiguous with an oligonucleotide sequence encoding a first amino acid linker sequence, contiguous with an oligonucleotide sequence encoding a sequence of amino acids identical to a human native B2M peptide sequence,
contiguous with an oligonucleotide sequence encoding a second amino acid linker sequence,
contiguous with an oligonucleotide sequence encoding a preselected second peptide sequence,
contiguous with an oligonucleotide sequence encoding a third amino acid linker,
contiguous with an oligonucleotide sequence encoding a second B2M leader sequence,
contiguous with an oligonucleotide sequence encoding a sequence of amino acids identical to a MHC heavy chain,
contiguous with an oligonucleotide sequence encoding a fourth amino acid linker,
contiguous with an oligonucleotide sequence encoding a sequence of amino acids identical to an immunoglobulin Fc domain,
contiguous with an oligonucleotide sequence encoding a fifth linker,
contiguous with an oligonucleotide sequence encoding a mammalian transmembrane domain.

A method of identifying a T-cell epitope comprising

contacting a T-cell with a plurality of isolated suspension-adapted cells comprising at least two cells, or a membrane-bound portion of such cells expressing the expression product, each cell or membrane bound portion expressing an expression product of a heterologous nucleic acid transduced or transfected therein, each of which expression products comprises a 5 to 20 amino acid peptide, contiguous with first linker peptide sequence, contiguous with a beta 2 microglobulin sequence, contiguous with a second linker peptide sequence, contiguous with a Major Histocompatibility Complex heavy chain sequence, contiguous with a third linker peptide sequence, contiguous with a fluorescent protein or an immunoglobulin Fc domain, contiguous with a fourth linker peptide sequence, contiguous with a mammalian transmembrane domain, wherein the plurality of isolated suspension-adapted cells or membrane-bound portions expresses at least two different encoded 5 to 20 amino acid peptides among the cells or membrane-bound portions under conditions permitting T-cells to conjugate with the 5 to 20 amino acid peptides;
recovering T-cell(s) which have formed a conjugate with a suspension-adapted cell or membrane-bound portions;
recovering DNA from the suspension-adapted cell(s);
sequencing the recovered DNA;
identifying the 5 to 20 amino acid peptide(s) encoded for in the DNA,
so as to thereby identify a T-cell epitope.

A method of identifying a T-cell epitope comprising

contacting a T-cell with a plurality of isolated suspension-adapted cells comprising at least two cells, or membrane-bound portions thereof, each expressing an expression product of a heterologous nucleic acid transduced or transfected therein, each of which expression products comprises (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence,
wherein (i) is bound by one, or more than one, disulfide bond to (ii) a sequence of amino acids having the sequence of a MHC heavy chain contiguous with a fourth amino acid linker sequence contiguous with a sequence of amino acids identical to an immunoglobulin Fc domain contiguous with a fifth amino acid linker, contiguous with a mammalian transmembrane domain, wherein the plurality of isolated suspension-adapted cells or membrane-bound portions thereof expresses at least two different encoded 5 to 20 amino acid peptides among the cells or portions under conditions permitting T-cells to conjugate with the 5 to 20 amino acid peptides;
recovering T-cell(s) which have formed a conjugate with a suspension-adapted cell or membrane-bound portion;
recovering DNA from the suspension-adapted cell(s);
sequencing the recovered DNA;
   identifying the 5 to 20 amino acid peptide(s) encoded for in the DNA, so as to thereby identify a T-cell epitope.

A method of identifying a T-cell epitope comprising

contacting a T-cell with a plurality of isolated suspension-adapted cells comprising at least two cells, or virus-like particle or viruses associated with a membrane portion of such cells, the cells or membrane bound portion expressing an expression product of a heterologous nucleic acid transduced or transfected therein, each of which expression products comprises a 5 to 20 amino acid peptide, contiguous with first linker peptide sequence, contiguous with a beta 2 microglobulin sequence, contiguous with a second linker peptide sequence, contiguous with a Major Histocompatibility Complex heavy chain sequence, contiguous with a third linker peptide sequence, contiguous with a fluorescent protein or an immunoglobulin Fc domain, contiguous with a fourth linker peptide sequence, contiguous with a mammalian transmembrane domain, contiguous with a viral packaging sequence, wherein the plurality of isolated suspension-adapted cells or of virus-like particles or viruses associated with the membrane portion of the cells, expresses at least two different encoded 5 to 20 amino acid peptides among the cells or virus-like particles or viruses under conditions permitting T-cells to conjugate with the 5 to 20 amino acid peptides;
recovering T-cell(s) which have formed a conjugate with a suspension-adapted cell, virus-like particle or virus of the plurality;
recovering DNA from the suspension-adapted cell or RNA from the virus-like particle or virus;
sequencing the recovered DNA or RNA;
identifying the 5 to 20 amino acid peptide(s) encoded for in the DNA or RNA,
   so as to thereby identify a T-cell epitope.

A method of identifying a T-cell epitope comprising

contacting a T-cell with a plurality of isolated suspension-adapted cells comprising at least two cells, or virus-like particles or viruses associated with a membrane portion of such a cells, each expressing an expression product of a heterologous nucleic acid transduced or transfected therein, each of which expression products comprises (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence,
wherein (i) is bound by one, or more than one, disulfide bond to (ii) a sequence of amino acids having the sequence of a MHC heavy chain contiguous with a fourth amino acid linker sequence contiguous with a sequence of amino acids identical to an immunoglobulin Fc domain contiguous with a fifth amino acid linker, contiguous with a mammalian transmembrane domain, wherein the plurality of isolated suspension-adapted cells, virus-like particles or viruses expresses at least two different encoded 5 to 20 amino acid peptides among the cells thereof under conditions permitting T-cells to conjugate with the 5 to 20 amino acid peptides;
recovering T-cell(s) which have formed a conjugate with a suspension-adapted cell, virus-like particle or virus associated membrane portion;
recovering DNA from the suspension-adapted cell or RNA from the virus-like particle or virus;
sequencing the recovered DNA or RNA;
identifying the 5 to 20 amino acid peptide(s) encoded for in the DNA or RNA, so as to thereby identify a T-cell epitope.

### BRIEF DESCRIPTION OF THE DRAWINGS

FIG. 1. General overview of the “epiCELL” immunomonitoring platform for high-throughput identification of CD8⁺ T-cell epitopes. A library of sc-pMHC vectors is pooled and transfected en masse into suspension adapted HEK293 cells, generating the epiCELL pool. The pooled expression library is mixed with patient derived peripheral T-cells and allowed to form conjugates, which are recovered by magnetic separation or more traditional flow cytometric sorting procedures. Magnetic beads, if used, can be from any commercial source, including Dynabeads® CD8 from Life technologies and CD8 microbeads (MACS) from Miltenyi. For example, superparamagnetic beads coupled with an anti-human CD8 antibody that enable easy isolation or depletion of human CD8+ T cells directly from any sample, including whole blood, bone marrow, buffy coat, mononuclear cells (MNC), and tissue digests. The epitope sequences from the enriched pool members are amplified by PCR using universal primers and subjected to next-generation deep sequencing to identify epitopes enriched by the capture process.

FIG. 2. Design of membrane-anchored class I sc-pMHC construct. Construct utilizes a native human B2M leader sequence immediately followed by a candidate epitope (labeled as peptide), further coupled to the native B2M molecule, the human HLA-A02:01, and a surface exposed mCherry expression proxy through linker regions (4 repeats of GGGGS for each of the first second and third linkers and optionally a 2 repeat GGGGS for as a fourth linker between the fluorescent protein and HC TM). The entire construct is held in the membrane through a native Class-I Heavy Chain transmembrane domain (HC TM). Universal primers (labeled Forward, Reverse) are used to amplify the unique 27-nucleotide sequence (9-mer peptide) following T-cell challenge to directly identify disease relevant epitopes.

FIG. 3. Surface expression validation of MHC controls. Expression validation of 4 known pathogenic HLA-A02 restricted epitopes linked to 4 independent viral pathogens displayed in our sc-pMHC epiCELL platform. The constructs being examined are CMV pp65 protein residues 495-504 [CMV], Influenza matrix protein 58-66 [FLU],], HTLV Tax 11-19 [HTLV] and HIV gag p17 76-84 [HIV]. Surface expression of constructs validated through fluorescence activated cell sorting (FACS) analysis monitoring mCherry proxy expression and anti-mCherry surface expression. Notably, subsequent to the generation of this figure an additional (5^{th}) control epitope was identified and added to test set encoding for EBV BMLF1 residues 259-267 [EBV]. The surface expression profile of EBV mirrors those observed for the other 4 controls (data not shown).

FIG. 4. Validating proper folding and epitope presentation of the MHC controls. Here, plasma membrane surface staining of Class-I HLA:B2M complexes is shown using W6/32 anti-MHC-Class I mAb illustrating 1) endogenous expression of Class I MHC (labeled as Parental), 2) ˜95% knock down through lentivral delivery of shRNA targeting the 5′ UTR of native human B2M (Knock-Down), and 3) rescue of Class-I MHC expression upon addition of our sc-p construct (Rescue). Notably, the construct does not contain the 5′ UTR and thus is immune to shRNA down-regulation. The mAb W6/32 used requires that both MHC and B2M are properly folded and membrane localized for binding.

FIG. 5. Constructs used for TCR expression and membrane localization in HEK cells. To allow for expression of control TCRs lentiviral co-transduction techniques were used, wherein one lentiviral construct harbors the full CD3 gene cassette (top) linked by various viral 2A peptides. The 2A “self-cleaving” peptides used were derived from the foot-and-mouth disease virus (F2A), Thosea asigna virus (T2A) and the equine rhinitis A virus (E2A). The second construct (bottom) carries the TCR alpha and beta chains linked by a viral porcine teschovirus-1 (P2A) peptide to allow for stoichiometric expression of each chain as this peptide shows the highest “cleavage” efficiency in mammalian cells [2]. The mCerulean (BLUE) expression proxy follows the beta chain transmembrane segment.

FIGS. 6A-6F. Surface expression of active hetero-dimeric TCR control constructs in HEK cells Proof-of-principle studies employ the 5 cognate TCRs for the HLA molecules discussed above (TCR RA14 [binds to CMV peptide], JM22 [FLU], AS01 [EBV], A06 [HTLV] and 1803 [HIV]). Surface expression and active T-cell complex formation confirmed through FACS analysis against surface anti-CD3 (FITC labeled, x-axis) and surface MHC pentamer staining (Phycoerythrin [PE], y-axis). Untransduced cells were used as a negative control (CNTRL).

FIGS. 7A-7G. Arrayed cell-cell FACS analysis for Initial validation of the epiCELL platform. The 5 TCRs (RA14, JM22, ASO1, 1803, A06) were individually expressed to complement the 5 cognate sc-pMHC epiCELLS (CMV, FLU, EBV, HIV, HTLV). Cytoplasmic mCherry (CYTO) and surface expressed mCherry (without the MHC, STALK) were used as negative controls. Histograms from FACS analysis of the individual and mixed populations clearly demonstrated a significant increase (as much as 100-fold, A06:HTLV interaction) in signal representing specific cell-cell interactions only when cells expressing cognate MHC:TCR pairs were both present, and correlate with traditional pentamer challenge (FIG. 6). Positive interactions are marked with red arrows.

FIG. 8. Results for the epiCELL platform. Two epiCELL constructs (CMV and FLU) were pooled, challenged with independent TCR bearing HEK cells (JM22), and sorted on the conjugates formed (using the mCherry surface expression proxy to track the epiCELL and mCerulean as the TCR expression proxy). (The fluorescent protein is not a required part of the construct when magnetic separation is being used, but is helpful for manual or lower throughput processes). The genomic DNA from each pool was extracted and subjected to ˜30 cycles of PCR using universal primers targeting flanking regions around the epitope. The resulting PCR bands are shown (top) for the Pre-sorted epiCELL pool (labeled as Pre) and JM22 challenged sets. These amplicons were submitted for library preparation and subsequent next generation sequencing was performed on an illumina MiSeq platform. The sequencing files were analyzed and epitopes readily identified. For each, the absolute number of epitope sequences observed were counted and normalized as a percent of ALL observed NGS reads passing our QC filter, the labels represent the pathogenic epitope for CMV and FLU (bottom).

FIG. 9. Five epiCELL constructs (CMV, FLU, EBV, HIV, HTLV) were pooled, challenged with independent TCR-bearing HEK cells (R14, JM22, AS01, A06), and sorted on the conjugates formed (using the mCherry surface expression proxy to track the epiCELL and mCerulean as the TCR expression proxy). The genomic DNA from each pool was extracted and subjected to ˜30 cycles of PCR using universal primers targeting flanking regions around the epitope. The bioanlyzer output for the resulting PCR bands are shown (top) for the Pre-sorted epiCELL pool (labeled as Pre) and TCR challenged sets. These amplicons were submitted for library preparation and subsequent next generation sequencing was performed on an illumina MiSeq platform. The sequencing files were analyzed and epitopes readily identified. Epitopes identified within the pre-sorted population (the library) was within a range from 16-23% (data not shown). For each of the TCR challenged data sets, the absolute number of epitope sequences observed were counted and normalized as a percent of all observed NGS reads that pass our QC filter and was used to calculate a Z-score.

FIG. 10A-10B. Exemplary alternate surface expression constructs for use in epiCELL and its derivatives, e.g., viratope. The variants utilize a native human B2M leader sequence immediately followed by a candidate epitope (labeled as peptide) further coupled to the B2M molecule through linker L1. 10A: A(1) is analogous to a “traditional” epiCELL based presentation with the addition of a viral packaging signal at the extreme C-terminal end (e.g, GP41 env residues 706-713, etc., labeled as VP). To allow for bivalent display, an Fc Fusion based construction has been utilized A(2), again terminating in a VP packaging signal. In this instance, epitopes for traditional antibodies (e.g., FLAG, MYC, etc.) are placed in linker L4 to allow for surface detection. Lastly, to increase the modularity/flexibility of the epiCELL screening platform, synTac based expression constructs are utilized. synTac's split the MHC construct into respective heavy and light chains and fuse both peptides and proteins to various ends (e.g., construct A(3) and schematically represented in panel 10B). All components associate during production within eukaryotic cells (e.g., HEK, CHO) and self-assemble. Individual chains are covalently tethered through disulfide bridges (shown as RED lines). All constructs are held in the membrane through a native Class-I Heavy Chain transmembrane domain (TM).

FIG. 11A-11B: RT-PCR and Next GEN Sequencing from viratope particle's. The genomic RNA from each viratope pool was extracted through lysis and subjected to one round of reverse transcription (RT, 42 degrees C. for 20 minutes), followed by ˜30 cycles of PCR using universal primers targeting flanking regions around the epitope. The resulting PCR bands are shown in panel 11A. Notably, a PCR band is only observed in the presence of an initial RT step (lane 1) and is absent when RT is omitted (lane 2), supporting the generation of competent retrovirus derived from epiCELLS. These amplicons were submitted for next generation sequencing (NGS) and epitopes readily identified (Panel 11B).

FIG. 12A-B: Viratope: lentiviral particles pseudotyped with peptide-HLA-A*0201 Fc fusion proteins for detection of antigen-specific T cell populations. Single chain constructs (FIG. 10A, No. 2) composed of a peptide epitope linked to beta-2 microglobulin, HLA-A*0201, and human IgG1 Fc were substituted for the envelope component of a third generation lentiviral transfection system. The constructs also contained a FLAG epitope tag for detection by secondary antibodies (placed in the L4 linker region). The peptide epitopes presented in the context of HLA-A*0201 were either the NLVPMVATV peptide epitope from human cytomegalovirus (CMV) or the GILGFVFTL peptide epitope from influenza (FLU). Harvested lentivirus was concentrated and applied to HEK cells previously transfected with either a specific or irrelevant T cell receptor (TCR). Excess lentivirus was washed from cells and the remaining cell-bound lentivirus was detected via a PE-conjugated anti-FLAG antibody. Lentivirus pseudotyped with the cognate, but not the irrelevant epitope bound to the respective cognate TCR-expressing HEK cells in a manner comparable to staining by specific peptide-MHC pentamers.

### DETAILED DESCRIPTION OF THE INVENTION

An isolated suspension-adapted cell is provided, wherein the cell is transduced by or transfected with a heterologous nucleic acid comprising, in 5′ to 3′ order:

a leader oligonucleotide sequence, contiguous with
an oligonucleotide sequence encoding a 5 to 20 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain sequence, contiguous with
an oligonucleotide sequence encoding a third linker, contiguous with
an oligonucleotide sequence encoding a fluorescent protein or encoding an immunoglobulin Fc domain, contiguous with
an oligonucleotide sequence encoding a fourth linker, contiguous with
an oligonucleotide sequence encoding a mammalian transmembrane domain.

In an embodiment of the cell, and of the other cells and constructs discussed herein comprising an immunoglobulin Fc domain, the immunoglobulin Fc domain can have the sequence of a

human Ig Fc, preferably a human IgG1 Fc. In another embodiment, such immunoglobulin Fc domain can have the sequence of a murine IgG2a Fc. Notably, where there are expressed constructs each comprising an immunoglobulin Fc domain, spontaneous bivalent fusion may occur. Accordingly, the discussed transduced or transfected cells, membrane-bound portions of such expressing the expression products as well as virus-like particles and viruses as described herein may demonstrate bivalent fusion of the expressed immunoglobulin Fc domains.

In an embodiment of the encoded, or the expressed, 5 to 20 amino acid peptides described herein, the peptide is one of 5, 6, 7, 8, 9, 10, 11, 12, 13, 14, 15, 16, 17, 18, 19 or 20 amino acids in length. In an embodiment the peptide is 8, 9, 10, 11, or 12 amino acids in length. In an embodiment the peptide is a nonamer (i.e. 9 amino acids in length). The sequence can be preselected as desired.

In an embodiment of the cell, and of the other cells and constructs discussed herein comprising a mammalian transmembrane domain, the transmembrane domain has the sequence of a mammalian transmembrane domain but is not taken from a mammal itself, for example it is a sequence engineered to have an identical or similar sequence to a mammalian transmembrane domain protein sequence. In an embodiment, the sequence is the same as a mammalian MHC transmembrane sequence. In an embodiment, the sequence is the same as a Major Histocompatibility Complex heavy chain transmembrane domain. In an embodiment, the sequence is the same as a Class I Major Histocompatibility Complex heavy chain transmembrane domain. MHC I alpha 3 sequences are known in the art. In an embodiment, the sequence is the same as a human Class I Major Histocompatibility Complex heavy chain transmembrane domain. As used herein, “contiguous with” in regard to two nucleotide sequences means the first sequence is consecutive with the second sequence via, for example, a phosphodiester bond. As used herein, “contiguous with” in regard to two peptide/oligopeptide sequences means the first sequence is consecutive with the second sequence via, for example, a peptide bond.

Any nucleic acid-encoded fluorescent proteins are usable in the invention described herein. Such proteins are well-known in the art. Non-limiting examples include a GFP, RFP, YFP, mFRUIT, mPlum, mCherry, tdTomato, mStrawberry, J-Red, DsRed-monomer, mOrange, mKO, mCitrine, Venus, YPet, EYFP, Emerald, EGFP, CyPet, mCFPm, Cerulean, and T-Sapphire.

A suspension-adapted cell is one that is able to survive or proliferate in a suspension culture. A heterologous nucleic acid is one that is heterologous relative to the cell into which it is transfected or transduced, the heterologous nucleic acid as a whole not naturally existing in the cell prior to transfection or transduction.

Linker sequences are short peptide sequences, including short repeat peptide sequences, known in the art. They generally do not interfere with or have minimal functional impact on other encoded peptide functions of the domains or regions they link. For example, a linker can be 4 repeats of GGGGS for one or more linker(s). Linkers as described herein, apart from the specific exception of the self-cleaving linker as referred to herein are stable in that they are not-self cleaving. With regard to the exception referred to, the self-cleaving linker, a non-limiting example of such is a viral P2A peptide, which peptides shows good self-cleaving efficiency in mammalian cells.

In an embodiment of the isolated suspension-adapted cell, the cell is transduced by or transfected with a heterologous nucleic acid comprising, in 5′ to 3′ order:

a leader oligonucleotide sequence, contiguous with
an oligonucleotide sequence encoding a 5 to 20 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain sequence, contiguous with
an oligonucleotide sequence encoding a third linker, contiguous with
an oligonucleotide sequence encoding an immunoglobulin Fc domain, contiguous with
an oligonucleotide sequence encoding a fourth linker, contiguous with
an oligonucleotide sequence encoding a mammalian transmembrane domain.

In an embodiment of the isolated suspension-adapted cell, the cell is transduced by or transfected with a heterologous nucleic acid comprising, in 5′ to 3′ order:

a leader oligonucleotide sequence, contiguous with
an oligonucleotide sequence encoding a 5 to 20 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain sequence, contiguous with
an oligonucleotide sequence encoding a third linker, contiguous with
an oligonucleotide sequence encoding a fluorescent protein, contiguous with
an oligonucleotide sequence encoding a fourth linker, contiguous with
an oligonucleotide sequence encoding a mammalian transmembrane domain.

Also provided is an isolated suspension-adapted cell expressing an expression product of a heterologous nucleic acid transduced or transfected therein, or a membrane-bound portion of such cell expressing the expression product, which expression product comprises, in N-terminal to C-terminal order:

a 5 to 20 amino acid peptide, contiguous with
a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a fluorescent protein or a sequence of an immunoglobulin Fc domain, contiguous with
a fourth linker peptide sequence, contiguous with
a mammalian transmembrane domain.

In an embodiment, the membrane-bound portion expressing the expression product of the cell is provided. In an embodiment, the membrane-bound portion is a microvessicle or a exosome.

In an embodiment, the cell expresses the expression product comprising the sequence of the immunoglobulin Fc domain.

The invention also provides the cell as described, or the membrane-bound portion, except wherein a linker thereof, such as a fourth linker, is additionally connected to a fluorescent protein (such as, for example, an mCherry) or an epitopes for a known antibodies (e.g., FLAG, MYC) as proxy for surface expression. In an embodiment, the linker of the cell as described, or the membrane-bound portion does not comprise such and is only a linker (for example as described elsewhere herein).

In an embodiment of the isolated suspension-adapted cell, or membrane-bound portion of such cell expressing the expression product, the cell expresses the expression product comprising the fluorescent protein.

In an embodiment of the isolated suspension-adapted cell, or membrane-bound portion of such cell expressing the expression product, the cell expresses the expression product comprising the immunoglobulin Fc domain.

In an embodiment of the isolated suspension-adapted cell, or membrane-bound portion of such cell expressing the expression product, the mammalian transmembrane domain is a Major Histocompatibility Complex heavy chain transmembrane domain.

In an embodiment, the heterologous nucleic acid further comprises an oligonucleotide encoding a viral packaging sequence 3′ relative to the oligonucleotide sequence encoding the mammalian transmembrane domain.

In an embodiment of the isolated suspension-adapted cell, or of the membrane-bound portion of such cell expressing the expression product, the expression product further comprises a viral packaging sequence that is C-terminal relative to the mammalian transmembrane domain.

In an embodiment of the transduced cells, recombinant nucleic acids, or heterologous nucleic acids described herein that encode a viral packaging sequence, the relevant nucleic acid can be, in an embodiment, an RNA sequence. In an embodiment, the viral packaging sequence is a retroviral viral packaging sequence.

In an embodiment, a membrane-bound portion expressing the expression product of the cell as described herein is provided, and is a viral like particle.

In an embodiment of the isolated suspension-adapted cells as described herein or the membrane-bound portion of such cells expressing the expression product, the beta 2 microglobulin has the same sequence as a human beta 2 microglobulin.

In an embodiment of the isolated suspension-adapted cells as described herein or the membrane-bound portion of such cells expressing the expression product, the Major Histocompatibility Complex heavy chain sequence has the same sequence as a human HLA-A sequence.

In an embodiment of the isolated suspension-adapted cells as described herein or the membrane-bound portion of such cells expressing the expression product, the transmembrane domain has the same sequence as a human Major Histocompatibility Complex I heavy chain transmembrane domain.

Also provided is a plurality of the isolated suspension-adapted cells as described. Also provided is a plurality of membrane-bound portions of such cells expressing the expression product, wherein the plurality comprises at least two different encoded 5 to 20 amino acid peptides.

In an embodiment of the pluralities, the plurality comprises at least 100 different encoded 5 to 20 amino acid peptides.

Also provided is the isolated suspension-adapted cell as described or membrane-bound portion of such cell expressing the expression product, or the plurality of the isolated suspension-adapted cells or membrane-bound portions of described, wherein the encoded peptide(s) is a nonamer or are nonamers. In an embodiment the encoded 5-20 amino acid peptide or peptides is or are presented on an extracellular surface of the cells.

Also provided is a membrane-bound portion of an isolated suspension-adapted cell as described expressing the expression product.

Also provided is an isolated suspension-adapted cell as described.

In an embodiment of the isolated suspension-adapted cell(s), the heterologous nucleic acid encodes the viral packaging sequence and the cell is transduced by or transfected with a virus, plasmid or viral vector comprising the heterologous nucleic acid.

Also provided is a (i) virus-like particle or (ii) virus, produced by the transduced or transfected cell as described herein, which virus like particle or a virus is physically associated via a cell membrane portion having attached thereto, by a mammalian transmembrane domain, an expression product comprising in N-terminal to C-terminal order:

a 5 to 20 amino acid peptide, contiguous with
a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a fluorescent protein or a sequence of an immunoglobulin Fc domain, contiguous with
a fourth linker peptide sequence, contiguous with
the mammalian transmembrane domain, contiguous with
a viral packaging sequence.

In an embodiment of the (i) virus-like particle or (ii) virus, the cell is transfected using a retroviral transfection system. In an embodiment of the (i) virus-like particle or (ii) virus, the transfection is effected using a lentiviral transfection system.

In an embodiment of the (i) virus-like particle or (ii) virus, the retroviral transfection system comprises a packaging plasmid having therein, in place of an oligonucleotide sequence or sequences encoding one or more envelope proteins, an oligonucleotide sequence or sequences encoding a 5 to 20 amino acid peptide, contiguous with

a first linker peptide sequence, contiguous with
a beta 2 microglobulin sequence, contiguous with
a second linker peptide sequence, contiguous with
a Major Histocompatibility Complex heavy chain sequence, contiguous with
a third linker peptide sequence, contiguous with
a fluorescent protein or an immunoglobulin Fc domain, contiguous with
a fourth linker peptide sequence, contiguous with
the mammalian transmembrane domain.

Also provided is an isolated virus, which virus has budded from the cell as described herein. Budded viruses take with them, or are associated with, a portion of the membrane of the cell and as such are associated with the expressed membrane located constructs described herein.

Also provided is an isolated virus-like particle has budded from the cell as described herein. Budded virus-like particles take with them, or are associated with, a portion of the membrane of the cell and as such are associated with the expressed membrane located constructs described herein.

In an embodiment, the virus is a retrovirus. In an embodiment, the virus is a lentivirus. In an embodiment, the retrovirus is recombinant.

Also provided is a plurality of the isolated viruses as described herein. In an embodiment, the plurality comprises viruses which differ in the encoded 5 to 20 amino acid peptides thereof.

Also provided is a plurality of the isolated virus-like particles as described herein. In an embodiment, the plurality comprises virus-like particles which differ in the encoded 5 to 20 amino acid peptides thereof.

In an embodiment of the viruses, the expressed recombinant polypeptide comprises the fluorescent protein. In an embodiment of the viruses, the expressed recombinant polypeptide comprises the immunoglobulin Fc domain. In an embodiment of the virus-like particles, the expressed recombinant polypeptide comprises the fluorescent protein. In an embodiment of the virus-like particles, the expressed recombinant polypeptide comprises the immunoglobulin Fc domain.

Also provided is a recombinant nucleic acid comprising, in 5′ to 3′ order:

a sequence encoding a leader oligonucleotide sequence, contiguous with
an oligonucleotide sequence encoding a 5 to 20 amino acid peptide, contiguous with
an oligonucleotide sequence encoding a first linker, contiguous with
an oligonucleotide sequence encoding a beta 2 microglobulin sequence, contiguous with
an oligonucleotide sequence encoding a second linker, contiguous with
an oligonucleotide sequence encoding a Major Histocompatibility Complex heavy chain sequence, contiguous with
an oligonucleotide sequence encoding a third linker peptide sequence, contiguous with
an oligonucleotide sequence encoding a fluorescent protein or an immunoglobulin Fc domain, contiguous with
an oligonucleotide sequence encoding a fourth linker peptide sequence, contiguous with
an oligonucleotide sequence encoding a mammalian transmembrane domain.

In an embodiment, the recombinant nucleic acid comprises the oligonucleotide sequence encoding the fluorescent protein. In an embodiment, the recombinant nucleic acid comprises the oligonucleotide sequence encoding the immunoglobulin Fc domain. In an embodiment, the mammalian transmembrane domain is a Major Histocompatibility Complex heavy chain transmembrane domain. In an embodiment, the mammalian transmembrane domain has the same sequence is a mammalian HLA-A*0201 domain. In an embodiment, the HLA-A*0201 is human.

In an embodiment, the recombinant nucleic acid further comprises an oligonucleotide encoding a viral packaging sequence 3′ relative to the oligonucleotide sequence encoding the mammalian transmembrane domain. In an embodiment, the recombinant nucleic acid is a vector. In an embodiment, the recombinant nucleic acid is a viral vector. In an embodiment, the recombinant nucleic acid is a retroviral vector. In an embodiment, the recombinant nucleic acid is a lentiviral vector. In an embodiment, the recombinant nucleic acid vector is a plasmid.

In an embodiment, of the recombinant nucleic acid or of the isolated suspension-adapted cells, or the heterologous or recombinant nucleic acid comprises cDNA.

Also provided is an isolated suspension-adapted cell transduced by or transfected with a heterologous nucleic acid comprising, in 5′ to 3′ order:

an oligonucleotide sequence encoding a first B2M leader sequence,
contiguous with an oligonucleotide sequence encoding a preselected 5 to 20 amino acid peptide, contiguous with an oligonucleotide sequence encoding a first amino acid linker sequence, contiguous with an oligonucleotide sequence encoding a sequence of amino acids identical to a human native B2M peptide sequence,
contiguous with an oligonucleotide sequence encoding a second amino acid linker sequence,
contiguous with an oligonucleotide sequence encoding a preselected second peptide sequence,
contiguous with an oligonucleotide sequence encoding a third amino acid linker,
contiguous with an oligonucleotide sequence encoding a second B2M leader sequence,
contiguous with an oligonucleotide sequence encoding a sequence of amino acids identical to a MHC heavy chain,
contiguous with an oligonucleotide sequence encoding a fourth amino acid linker,
contiguous with an oligonucleotide sequence encoding a sequence of amino acids identical to an immunoglobulin Fc domain,
contiguous with an oligonucleotide sequence encoding a fifth linker,
contiguous with an oligonucleotide sequence encoding a mammalian transmembrane domain.

In an embodiment, the preselected second peptide sequence is an immune system effector molecule. In an embodiment, the preselected second peptide sequence is a detectable epitope. In non-limiting examples the detectable epitope is a FLAG epitope or a MYC epitope. In an embodiment, the preselected second peptide sequence is a fluorescent protein, as described herein. In an embodiment the preselected second peptide sequence can be a naturally occurring or synthetic affinity reagent targeting, e.g., a cell surface glycan or other post-translational modification (e.g., sulfation). Examples include, but are not limited to, members of the TNF/TNFR family (OX40L, ICOSL, FASL, LTA, LTB TRAIL, CD153, TNFSF9, RANKL, TWEAK, TNFSF13, TNFSF13b, TNFSF14, TNFSF15, TNFSF18, CD40LG, CD70) or affinity reagents directed at the TNF/TNFR family members; members of the Immunoglobulin superfamily (VISTA, PD1, PD-L1, PDL2, B71, B72, CTLA4, CD28, TIM3, CD4, CD8, CD19, T cell receptor chains, ICOS, ICOS ligand, HHLA2, butyrophilins, BTLA, B7-H3, B7-H4, CD3, CD79a, CD79b, IgSF, CAMS including CD2, CD58, CD48, CD150, CD229, CD244, ICAM-1), Leukocyte immunoglobulin like receptors (LILR), killer cell immunoglobulin like receptors (KIR)), lectin superfamily members, selectins, cytokines/chemokine and cytokine/chemokine receptors, growth factors and growth factor receptors), adhesion molecules (integrins, fibronectins, cadherins), or ecto-domains of multi-span intergral membrane protein, or affinity reagents directed at the Immunoglobulin superfamily and listed gene products. In addition, active homologs/orthologs of these gene products, including but not limited to, viral sequences (e.g., CMV, EBV), bacterial sequences, fungal sequences, eukaryotic pathogens (e.g., Schistosoma, Plasmodium, Babesia, Eimeria, Theileria, Toxoplasma, Entamoeba, Leishmania, and trypanosoma), and mammalian-derived coding regions. In an embodiment the preselected second peptide sequence can be a T cell stimulatory domain or can be a T cell inhibitory domain. In an embodiment the preselected second peptide sequence can be cell surface protein ectodomain.

Also provided is an isolated suspension-adapted cell expressing an expression product of a heterologous nucleic acid transduced or transfected therein, or a membrane-bound portion of such cell expressing the expression product, which expression product comprises,

a recombinant polypeptide construct comprising (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence, wherein (i) is bound by one, or more than one, disulfide bond to (ii) a sequence of amino acids having the sequence of a MHC heavy chain contiguous with a fourth amino acid linker
sequence contiguous with a sequence of amino acids identical to an immunoglobulin Fc domain contiguous with a fifth amino acid linker, contiguous with a mammalian transmembrane domain. In embodiments, the preselected second peptide sequence, and the other components, are as recited elsewhere herein.

Also provided is an isolated suspension-adapted cell transduced by or transfected with a virus, plasmid or viral vector comprising a heterologous nucleic acid comprising, in 5′ to 3′ order:

an oligonucleotide sequence encoding a first B2M leader sequence,
contiguous with an oligonucleotide sequence encoding a preselected 5 to 20 amino acid peptide, contiguous with an oligonucleotide sequence encoding a first amino acid linker sequence, contiguous with an oligonucleotide sequence encoding a sequence of amino acids identical to a human native B2M peptide sequence,
contiguous with an oligonucleotide sequence encoding a second amino acid linker sequence,
contiguous with an oligonucleotide sequence encoding a preselected second peptide sequence,
contiguous with an oligonucleotide sequence encoding a third amino acid linker,
contiguous with an oligonucleotide sequence encoding a second B2M leader sequence,
contiguous with an oligonucleotide sequence encoding a sequence of amino acids identical to a MHC heavy chain,
contiguous with an oligonucleotide sequence encoding a fourth amino acid linker,
contiguous with an oligonucleotide sequence encoding a sequence of amino acids identical to an immunoglobulin Fc domain,
contiguous with an oligonucleotide sequence encoding a fifth linker,

contiguous with an oligonucleotide sequence encoding a mammalian transmembrane domain, contiguous with an oligonucleotide encoding a viral packaging sequence. Viral packaging sequences or signals are known in the art and are also described herein. In an embodiment, the third amino acid linker is self-cleaving after expression. Self-cleaving linkers are described herein, such as the viral P2A peptide.

Also provided is a (i) virus like particle or (ii) virus, produced by the instant cell, which virus like particle or a virus is physically associated via a cell membrane portion having attached thereto, by a mammalian transmembrane domain, an expression product comprising in N-terminal to C-terminal order:

a recombinant polypeptide construct comprising (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence, wherein (i) is bound by one, or more than one, disulfide bond to (ii) a sequence of amino acids having the sequence of a MHC heavy chain contiguous with a fourth amino acid linker sequence contiguous with a sequence of amino acids identical to an immunoglobulin Fc domain contiguous with a fifth amino acid linker, contiguous with a mammalian transmembrane domain, contiguous with a viral packaging sequence. In an embodiment of the (i) virus like particle or (ii) virus, the mammalian transmembrane domain is a Major Histocompatibility Complex heavy chain transmembrane domain. In an embodiment, the heterologous nucleic acid further comprises an oligonucleotide encoding a viral packaging sequence 3′ relative to the oligonucleotide sequence encoding the mammalian transmembrane domain. In an embodiment of the (i) virus like particle or (ii) virus, the preselected second peptide is a T Cell modulatory domain, an antibody epitope, a fluorescent protein, a nucleic acid binding protein or a comodulatory protein.

Also provided is a recombinant nucleic acid comprising, in 5′ to 3′ order:

an oligonucleotide sequence encoding a first B2M leader sequence,
contiguous with an oligonucleotide sequence encoding a preselected 5 to 20 amino acid peptide, contiguous with an oligonucleotide sequence encoding a first amino acid linker sequence, contiguous with an oligonucleotide sequence encoding a sequence of amino acids identical to a human native B2M peptide sequence,
contiguous with an oligonucleotide sequence encoding a second amino acid linker sequence,
contiguous with an oligonucleotide sequence encoding a preselected second peptide sequence,
contiguous with an oligonucleotide sequence encoding a third amino acid linker,
contiguous with an oligonucleotide sequence encoding a second B2M leader sequence,
contiguous with an oligonucleotide sequence encoding a sequence of amino acids identical to a MHC heavy chain,
contiguous with an oligonucleotide sequence encoding a fourth amino acid linker,
contiguous with an oligonucleotide sequence encoding a sequence of amino acids identical to an immunoglobulin Fc domain,
contiguous with an oligonucleotide sequence encoding a fifth linker,
contiguous with an oligonucleotide sequence encoding a mammalian transmembrane domain.

In an embodiment of the recombinant nucleic acid, the mammalian transmembrane domain is a Major Histocompatibility Complex heavy chain transmembrane domain. In an embodiment of the recombinant nucleic acid, the recombinant nucleic acid further comprises an oligonucleotide encoding a viral packaging sequence 3′ relative to the oligonucleotide sequence encoding the mammalian transmembrane domain.

Also provided is a method of identifying a T-cell epitope comprising

contacting a T-cell with a plurality of isolated suspension-adapted cells comprising at least two cells, or a membrane-bound portion of such cells expressing the expression product, each cell or membrane bound portion expressing an expression product of a heterologous nucleic acid transduced or transfected therein, each of which expression products comprises a 5 to 20 amino acid peptide, contiguous with first linker peptide sequence, contiguous with a beta 2 microglobulin sequence, contiguous with a second linker peptide sequence, contiguous with a Major Histocompatibility Complex heavy chain sequence, contiguous with a third linker peptide sequence, contiguous with a fluorescent protein or an immunoglobulin Fc domain, contiguous with a fourth linker peptide sequence, contiguous with a mammalian transmembrane domain, wherein the plurality of isolated suspension-adapted cells or membrane-bound portions expresses at least two different encoded 5 to 20 amino acid peptides among the cells or membrane-bound portions under conditions permitting T-cells to conjugate with the 5 to 20 amino acid peptides;
recovering T-cell(s) which have formed a conjugate with a suspension-adapted cell or membrane-bound portions;
recovering DNA from the suspension-adapted cell(s);
sequencing the recovered DNA;
identifying the 5 to 20 amino acid peptide(s) encoded for in the DNA,
so as to thereby identify a T-cell epitope.

In an embodiment of recovering the T-cells in the methods described herein, the conjugate is recovered.

Also provided is a method of identifying a T-cell epitope comprising contacting a T-cell with a plurality of isolated suspension-adapted cells comprising at least two cells, or membrane-bound portions thereof, each expressing an expression product of a heterologous nucleic acid transduced or transfected therein, each of which expression products comprises (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence, wherein (i) is bound by one, or more than one, disulfide bond to (ii) a sequence of amino acids having the sequence of a MHC heavy chain contiguous with a fourth amino acid linker

sequence contiguous with a sequence of amino acids identical to an immunoglobulin Fc domain contiguous with a fifth amino acid linker, contiguous with a mammalian transmembrane domain, wherein the plurality of isolated suspension-adapted cells or membrane-bound portions thereof expresses at least two different encoded 5 to 20 amino acid peptides among the cells or portions under conditions permitting T-cells to conjugate with the 5 to 20 amino acid peptides;
recovering T-cell(s) which have formed a conjugate with a suspension-adapted cell or membrane-bound portion;
recovering DNA from the suspension-adapted cell(s);
sequencing the recovered DNA;
identifying the 5 to 20 amino acid peptide(s) encoded for in the DNA,
so as to thereby identify a T-cell epitope.

In an embodiment of the methods, the T-cell(s) which have formed a conjugate are recovered by flow cytometry. In an embodiment of the methods, the T-cell(s) which have formed a conjugate are recovered by fluorescence activated cell sorting.

In an embodiment of the methods, the method comprises amplifying the recovered DNA prior to sequencing. In an embodiment of the methods, the amplifying is effected using one or more universal primers. In an embodiment of the methods, one or more of the universal primers is directed to a portion of the sequence of the heterologous nucleic acid but is not complementary to a nucleic acid encoding a native beta 2 microglobulin sequence of the cell.

In an embodiment of the methods, the mammalian transmembrane domain is a Major Histocompatibility Complex heavy chain transmembrane domain. In an embodiment of the methods, the T-cells comprise peripheral T-cells obtained from a subject. In an embodiment of the methods, the subject is human.

In an embodiment of the methods, the method further comprises identifying any of the 5-20 amino acid peptides encoded for in the DNA that are enriched in the recovered DNA relative to their presence in the DNA of the plurality of isolated suspension-adapted cells, so as to thereby identify one or more immunodominant T-cell epitope(s).

In an embodiment of the methods, the methods further comprise comparing the level of the T cell conjugate with a level of control which is a recombinantly engineered T cell receptor (TCR)-expressing control cell, and wherein levels in excess of control indicate an immunodominant epitope.

In an embodiment of the methods, the isolated suspension-adapted cell is a mammalian cell. In an embodiment, the isolated suspension-adapted cell is an HEK cell.

In an embodiment of the methods, the isolated suspension-adapted cells are employed. In an embodiment of the methods, the isolated membrane-bound portions of the cells expressing the expression product suspension-adapted cells are employed.

Also provided is a method of identifying a T-cell epitope comprising

contacting a T-cell with a plurality of isolated suspension-adapted cells comprising at least two cells, or virus-like particle or viruses associated with a membrane portion of such cells, the cells or membrane bound portion expressing an expression product of a heterologous nucleic acid transduced or transfected therein, each of which expression products comprises a 5 to 20 amino acid peptide, contiguous with first linker peptide sequence, contiguous with a beta 2 microglobulin sequence, contiguous with a second linker peptide sequence, contiguous with a Major Histocompatibility Complex heavy chain sequence, contiguous with a third linker peptide sequence, contiguous with a fluorescent protein or an immunoglobulin Fc domain, contiguous with a fourth linker peptide sequence, contiguous with a mammalian transmembrane domain, contiguous with a viral packaging sequence, wherein the plurality of isolated suspension-adapted cells or of virus-like particles or viruses associated with the membrane portion of the cells, expresses at least two different encoded 5 to 20 amino acid peptides among the cells or virus-like particles or viruses under conditions permitting T-cells to conjugate with the 5 to 20 amino acid peptides;

recovering T-cell(s) which have formed a conjugate with a suspension-adapted cell, virus-like particle or virus of the plurality;

recovering DNA from the suspension-adapted cell or RNA from the virus-like particle or virus;

sequencing the recovered DNA or RNA;

identifying the 5 to 20 amino acid peptide(s) encoded for in the DNA or RNA,

so as to thereby identify a T-cell epitope.

Also provided is a method of identifying a T-cell epitope comprising

contacting a T-cell with a plurality of isolated suspension-adapted cells comprising at least two cells, or virus-like particles or viruses associated with a membrane portion of such cells, each expressing an expression product of a heterologous nucleic acid transduced or transfected therein, each of which expression products comprises (i) a preselected 5 to 20 amino acid peptide bound by a first amino acid linker sequence contiguous with a sequence of amino acids comprising a sequence identical to a human native B2M peptide sequence contiguous with a second amino acid linker sequence contiguous with a preselected second peptide sequence,

wherein (i) is bound by one, or more than one, disulfide bond to (ii) a sequence of amino

acids having the sequence of a MHC heavy chain contiguous with a fourth amino acid linker

sequence contiguous with a sequence of amino acids identical to an immunoglobulin Fc

domain contiguous with a fifth amino acid linker, contiguous with a mammalian transmembrane domain, wherein the plurality of isolated suspension-adapted cells, virus-like particles or viruses expresses at least two different encoded 5 to 20 amino acid peptides among the cells thereof under conditions permitting T-cells to conjugate with the 5 to 20 amino acid peptides;

recovering T-cell(s) which have formed a conjugate with a suspension-adapted cell, virus-like particle or virus associated membrane portion;

recovering DNA from the suspension-adapted cell or RNA from the virus-like particle or virus;

sequencing the recovered DNA or RNA;

identifying the 5 to 20 amino acid peptide(s) encoded for in the DNA or RNA,

so as to thereby identify a T-cell epitope.

In an embodiment, the virus-like particles or viruses associated with a membrane portion of the cell have budded from such cells.

In an embodiment of the methods, the T-cell(s) which have formed a conjugate are recovered by flow cytometry. In an embodiment of the methods, the T-cell(s) conjugates are recovered via FACS or secondary antibody staining methods. In an embodiment, the secondary antibody is directed to a preselected second peptide sequence.

In an embodiment the methods comprise amplifying the recovered DNA or RNA prior to sequencing. In an embodiment, the amplifying is effected using one or more universal primers.

In an embodiment, the T-cell(s) which have formed a conjugate are recovered by (i) contacting the T-cell(s) which have formed a conjugate with a magnetic bead having attached to an external surface thereof an antibody or antibody fragment directed against a T-cell surface marker molecule and (ii) applying a magnetic field to the beads so as to recover the magnetic beads.

In an embodiment, the T-cell surface marker molecule is a CD8 molecule.

In an embodiment of the methods, the suspension adapted cells or membrane portions thereof express the fluorescent protein and T-cell(s) which have formed a conjugate are recovered by fluorescence activated cells sorting based on fluorescence of said fluorescent protein.

In an embodiment of the methods, the virus like particles or viruses are recovered by a secondary antibody-based system, wherein the secondary antibody is directed to an epitope in the expressed construct. Non-limiting examples of such epitopes include FLAG and MYC epitopes.

In an embodiment of the inventions described, isolated suspension-adapted cell, the beta 2 microglobulin has the same sequence as a human beta 2 microglobulin. In an embodiment of the isolated suspension-adapted cell, the Histocompatibility Complex heavy chain sequence has the same sequence as a human HLA-A sequence. In an embodiment of the isolated suspension-adapted cell, the Histocompatibility Complex heavy chain transmembrane domain has the same sequence as a human Major Histocompatibility Complex I heavy chain transmembrane domain

In an embodiment of the inventions described, the pluralities can comprises at least 100 different encoded 5-20 amino acid peptides. In an embodiment, the peptides are 8, 9, 10, 11 or 12 amino acid peptides. In an embodiment, the plurality comprises at least 1000 different encoded 8, 9, 10, 11 or 12 amino acid peptides. In an embodiment, the plurality comprises at least 10,000 different encoded 8, 9, 10, 11 or 12 amino acid peptides. In an embodiment, the plurality comprises at least 100,000 different encoded 8, 9, 10, 11 or 12 amino acid peptides. In an embodiment, the plurality comprises at least 1×10⁶ different encoded 8, 9, 10, 11 or 12 amino acid peptides. In an embodiment, the plurality comprises at least 1×10⁷ different encoded 8, 9, 10, 11 or 12 amino acid peptides. In an embodiment, the plurality comprises at least 1×10⁸ different encoded 8, 9, 10, 11 or 12 amino acid peptides.

In an embodiment of the isolated suspension-adapted cell, or of the plurality of the isolated suspension-adapted cells, the encoded peptide is a nonamer (9 amino acids in length).

In an embodiment of the inventions described, the encoded peptide is presented on an extracellular surface of the cells.

In an embodiment, the recombinant nucleic acid is a vector. In an embodiment, the vector is a viral vector. In an embodiment, the viral vector is a lentiviral vector.

In an embodiment of the isolated suspension-adapted cells, of the plurality of the isolated suspension-adapted cells, or of the recombinant nucleic acid, the nucleic acid comprises DNA.

In an embodiment, one or more of the universal primers is directed to a portion of the sequence of the heterologous nucleic acid but is not complementary to a nucleic acid encoding a native beta 2 microglobulin sequence of the cell.

In an embodiment, the T-cells comprise peripheral T-cells obtained from a subject.

In an embodiment of the methods herein, the subject is human.

In an embodiment, the method comprises comparing results obtained to those for a recombinantly engineered TCR-expressing control cell. In an embodiment, the recombinantly engineered TCR-expressing control cell is an HEK cell.

In an embodiment of the cells, the beta 2 microglobulin has the same sequence as a human beta 2 microglobulin. In an embodiment, the Histocompatibility Complex heavy chain sequence has the same sequence as a human HLA-A sequence. In an embodiment, the Histocompatibility Complex heavy chain transmembrane domain has the same sequence as a human Major Histocompatibility Complex I heavy chain transmembrane domain

A plurality of the isolated suspension-adapted cells is provided, wherein the plurality comprises at least two different encoded 8, 9, 10, 11 or 12 amino acid peptides.

In the context of isogenic cell lines (single integration per cell) the practical limit is equal to the complexity of the library used. In other words, scaled based on number of cells in the reaction—for example 10⁶-10⁸.

In an embodiment of the invention, the linker between the beta 2 microglobulin and the Major Histocompatibility Complex heavy chain can be removed resulting in two separate products. These will assemble naturally in the cell.

In an embodiment, the nucleic acid comprises the following sequence: atgtctcgctccgtggccttagctgtgctcgcgctactctctctttctggcctggaggcc(n)ₓggtggaggtggttctggaggaggc ggttcgggcggaggtggtagtatccagcgtactccaaagattcagghtactcacgtcatccagcagagaatggaaagtcaaatttcc tgaattgctatgtgtctgggtttcatccatccgacattgaagttgacttactgaagaatggagagagaattgaaaaagtggagcattcag acttgtctttcagcaaggactggtctttctatctcttgtattatactgaattcacccccactgaaaaagatgagtatgcctgccgtgtgaac cacgtgactttgtcacagcccaagatagttaagtgggatcgagacatgggaggcggaggatctggtggtggaggttctggtggtgg gggatctggctctcactccatgaggtatttcttcacatccgtgtcccggcccggccgcggggagccccgcttcatcgcagtgggcta cgtggacgacacgcagttcgtgcggttcgacagcgacgccgcgagccagaggatggagccgcgggcgccgtggatagagcag gagggtccggagtattgggacggggagacacggaaagtgaaggcccactcacagactcaccgagtggacctggggaccctgcg cggcgcctacaaccagagcgaggccggttctcacaccgtccagaggatgtatggctgcgacgtggggtcggactggcgcttcctc cgcgggtaccaccagtacgcctacgacggcaaggattacatcgccctgaaagaggacctgcgctcttggaccgcggcggacatg gcagctcagaccaccaagcacaagtgggaggcggcccatgtggcggagcagttgagagcctacctggagggcacgtgcgtgga gtggctccgcagatacctggagaacgggaaggagacgctgcagcgcacggacgcccccaaaacgcatatgactcaccacgctg tctctgaccatgaagccaccctgaggtgctgggccctgagcttctaccctgcggagatcacactgacctggcagcgggatgggga ggaccagacccaggacacggagctcgtggagaccaggcctgcaggggatggaaccttccagaagtgggcggctgtggtggtgc cttctggacaggagcagagatacacctgccatgtgcagcatgagggtttgcccaagcccctcaccctgagatgggagccgggtgg aggcggatctggcggcggaggatctggaggaggtggatctgggggcggtggtagtggcctgaatgacatctttgaagcccagaa aatcgaatggcacgaaatggtgagcaagggcgaggaggataacatggccatcatcaaggagttcatgcgcttcaaggtgcacatg gagggctccgtgaacggccacgagttcgagatcgagggcgagggcgagggccgcccctacgagggcacccagaccgccaag ctgaaggtgaccaagggtggccccctgcccttcgcctgggacatcctgtcccctcagttcatgtacggctccaaggcctacgtgaag caccccgccgacatccccgactacttgaagctgtccttccccgagggcttcaagtgggagcgcgtgatgaacttcgaggacggcg gcgtggtgaccgtgacccaggactcctccctccaggacggcgagttcatctacaaggtgaagctgcgcggcaccaacttcccctcc gacggccccgtaatgcagaagaagacaatgggctgggaggcctcctccgagcggatgtaccccgaggacggcgccctgaagg gcgagatcaagcagaggctgaagctgaaggacggcggccactacgacgctgaggtcaagaccacctacaaggccaagaagcc cgtgcagctgcccggcgcctacaacgtcaacatcaagttggacatcacctcccacaacgaggactacaccatcgtggaacagtac gaacgcgccgagggccgccactccaccggcggcatggacgagctgtacaagggtggaggtggttctggaggaggcggttcga gcagccagccgaccattccgattgtgggcattattgcgggcctggtgctgtttggcgcggtgattaccggcgcggtggtggcggcg gtgatgtggcgtcgtaaaagcagcgatcgtaaagattataaagatgatgatgataaataatag (SEQ ID NO:1), wherein (n)ₓ is an 8, 9, 10, 11 or 12 amino acid-encoding nucleotide sequence, with x being 24, 27, 30, 33, or 36 nucleotides, respectively. In an embodiment, the 24, 27, 30, 33, or 36 nucleotides are comprised of 8, 9, 10, 11, or 12 codons, or equivalents, respectively.

In an embodiment, the recombinant nucleic acid is up to 3000 nt for lentiviral delivery. In an embodiment, the recombinant nucleic acid is up to 10,000 nt for plasmid delivery.

In an embodiment, the nucleic encodes, or the expression product comprises, the following sequence: MSRSVALAVLALLSLSGLEAX₍ₙ₎GGGGSGGGGSGGGGSIQRTPKIQVYSRHPAENGKSNFLNCY VSGFHPSDIEVDLLKNGERIEKVEHSDLSFSKDWSFYLLYYTEFTPTEKDEYACRVNHVTLSQPK IVKWDRDMGGGGSGGGGSGGGGSGSHSMRYFFTSVSRPGRGEPRFIAVGYVDDTQFVRFDSDA ASQRMEPRAPWIEQEGPEYWDGETRKVKAHSQTHRVDLGTLRGAYNQSEAGSHTVQRMYGCD VGSDWRFLRGYHQYAYDGKDYIALKEDLRSWTAADMAAQTTKHKWEAAHVAEQLRAYLEG TCVEWLRRYLENGKETLQRTDAPKTHMTHHAVSDHEATLRCWALSFYPAEITLTWQRDGEDQ TQDTELVETRPAGDGTFQKWAAVVVPSGQEQRYTCHVQIIEGLPKPLTLRWEPGGGGSGGGGS GGGGSGGGGSGLNDIFEAQKIEWHEMVSKGEEDNMAIIKEFMRFKVHMEGSVNGHEFEIEGEG EGRPYEGTQTAKLKVTKGGPLPFAWDILSPQFMYGSKAYVKHPADIPDYLKLSFPEGFKWERV MNFEDGGVVTVTQDSSLQDGEFIYKVKLRGTNFPSDGPVMQKKTMGWEASSERMYPEDGALK GEIKQRLKLKDGGHYDAEVKTTYKAKKPVQLPGAYNVNIKLDITSHNEDYTIVEQYERAEGRHS TGGMDELYKGGGGSGGGGSSSQPTIPIVGIIAGLVLFGAVITGAVVAAVMWRRKSSDRKDYKD DDK (SEQ ID NO:2), wherein X₍ₙ₎ is a 8, 9, 10, 11, or 12 amino acid peptide sequence. (n) can be any one of 8, 9, 10, 11, or 12 or the range or a sub-range thereof.

A leader sequence includes any signal peptide that can be processed by a mammalian cell. Such sequences are well-known in the art.

Fluorescent proteins usable in the invention include GFP, RFP, YFP, mFRUIT. Any nucleic acid-encodable fluorescent protein may be used, for example mPlum, mCherry, tdTomato, mStrawberry, J-Red, DsRed-monomer, mOrange, mKO, mCitrine, Venus, YPet, EYFP, Emerald, EGFP, CyPet, mCFPm, Cerulean, T-Sapphire, GFP.

An exemplary non-limiting B2M Leader is MSRSVALAVLALLSLSGLEA (SEQ ID NO:3).

An exemplary non-limiting B2M sequence is IQRTPKIQVYSRHPAENGKSNFLNCYVSGFHPSDIEVDLLKNGERIEKVEHSDLSFSK DWSFYLLYYTEFTPTEKDEYACRVNHVTLSQPKIVKWDRDM (SEQ ID NO:4).

An exemplary non-limiting MHC Heavy Chain sequence is GSHSMRYFFTSVSRPGRGEPRFIAVGYVDDTQFVRFDSDAASQRMEPRAPWIEQEG PEYWDGETRKVKAHSQTHRVDLGTLRGAYNQSEAGSHTVQRMYGCDV GSDWRF LRGYHQYAYDGKDYIALKEDLRSWTAADMAAQTTKHKWEAAHVAEQLRAYLEG TCVEWLRRYLENGKETLQRTDAPKTHMTHHAVSDHEATLRCWALSFYPAEITLTW QRDGEDQTQDTELVETRPAGDGTFQKWAAVVVPSGQEQRYTCHVQHEGLPKPLT LRWEP (SEQ ID NO:5).

An exemplary non-limiting immunoglobulin Fc Domain sequence is DKTHTCPPCPAPELLGGP SVFLFPPKPKDTLMISRTPEVTCVVVDVSHEDPEVKFNW YVDGVEVHNAKTKPREEQYNSTYRVVSVLTVLHQDWLNGKEYKCKVSNKALPAP IEKTISKAKGQPREPQVYTLPPSREEMTKNQVSLTCLVKGFYPSDIAVEWESNGQPE NNYKTTPPVLDSDGSFFLYSKLTVDKSRWQQGNVFSCSVMHEALHNHYTQKSLSL SPGK (SEQ ID NO:6).

An exemplary non-limiting viral packaging (VP) sequence (signal) is NRVRQGYS (SEQ ID NO:7). In one embodiment, the viral packaging sequence is 8 to 20 amino acids in length. In one embodiment, the viral packaging sequence is 8 amino acids in length.

In one embodiment, the non-cleaving linkers are each, independently, from 5 to 40 amino acids in length. In one embodiment, the non-cleaving linkers are each, independently, from 5 to 30 amino acids in length. In one embodiment, the non-cleaving linkers are each, independently, from 5 to 20 amino acids in length. In one embodiment, the non-cleaving linkers are each 20 amino acids in length.

As used herein, having the “same sequence” means having 95% or greater sequence similarity with the referenced sequence without preventing the established or known function of the reference sequence. In an embodiment, having the same sequence means having a sequence completely identical to the referenced sequence.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

Herein is described a novel mammalian cell display platform for the presentation of candidate T-cell epitopes (“epiCELL”) for high throughput T-cell epitope mapping from patient samples (immunomonitoring), preferably using highly sensitive and massively parallel next-generation sequencing as the readout.

The approach centers on the use of a novel membrane-anchored single chain peptide MHC (sc-pMHC) mammalian cell display platform to allow for the presentation of large numbers of T-cell epitopes in the context of class I MHC on the surface of, e.g., HEK cells. These expression pools are challenged with T-cells from, for example, healthy, infected, cured and immunized patients to identify those epitopes that are directly relevant to disease, treatment and neutralization, for, in a non-limiting example, category A-C pathogens. Immunodominant signatures identified from the pathogens can be assembled into a pathogen epitope collection for use as a rapid and portable diagnostic tool, with detection occurring directly from whole blood.

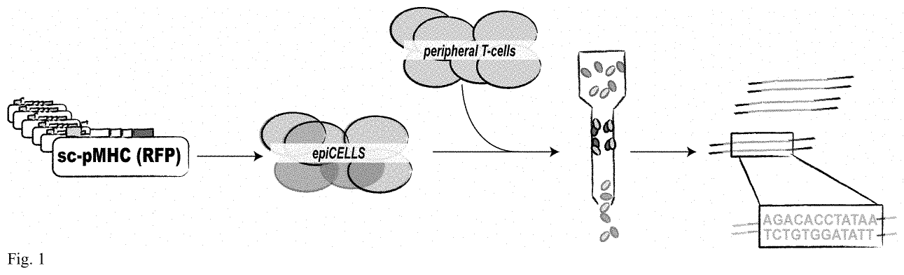

The preferred strategy exploits a library of sc-pMHC constructs displayed on the surface of mammalian cells and challenged against patient/cohort specific peripheral T-cells to directly identify disease relevant epitopes. As illustrated in FIG. 1, the library of sc-pMHC vectors can be pooled and transfected (or transduced in the case of, e.g., lentiviral pools) en masse into suspension adapted cells, such as HEK293 cells, thus generating an epiCELL pool.

### Example 1

The epiCELL pool is mixed with patient derived peripheral T-cells (purified from whole blood samples using standard protocols [26]) and allowed to form conjugates through the specific engagement of TCRs with their cognate sc-pMHC ligands expressed by the epiCELL pool. Conjugates are then recovered by, for example, magnetic separation for the processing of multiple patient samples in parallel, or more traditional flow cytometric sorting procedures for single samples. The epitope sequences from the enriched pool members are amplified by PCR (using universal primers) and subjected to next-generation deep sequencing to identify epitopes enriched by the capture process. These enriched epitopes directly identify immunodominant T-cell epitopes. Further subsequent validation, if desired, can be effected by in vitro methods (e.g. cytokine ELISpot, FACS) [27]. The strategy allows for the rapid identification of all disease relevant immunodominant epitopes from a single patient sample. Notably, this approach can be multiplexed through the use of indexed adapters, e.g. TruSeq®, to vastly increase throughput and reduce costs (e.g., multiple patient samples can be run on a single lane of an NGS flow cell).

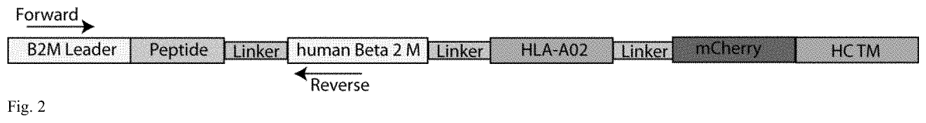

The Construct: One overall design for the membrane anchored class I sc-pMHC molecule is presented in FIG. 2. Briefly, this construct utilizes a native human B2M leader sequence to allow for plasma membrane localization immediately followed by a candidate epitope (labeled as peptide). Once in the ER the leader sequence is fully removed and allows for the presentation of the peptide in the MHC binding pocket. This is further coupled to the native B2M molecule, the human HLA-A02:01 allele, and a surface exposed mCherry expression proxy through linker regions (4 repeats of GGGGS (SEQ ID NO:8) for each linker). The entire construct is held in the membrane through a native Class-I Heavy Chain transmembrane domain (HC TM). The covalent linkage of antigenic peptides to the MHC class-I molecule is well established and has been highly effective both in vitro and in vivo [28-30]. This strategy eliminates difficulties associated with unintended T-cell binding/activation that arise from the exchange of non-covalent peptide complexes (cross-presentation), especially those that are weak binding. Furthermore, this modular design is readily amenable to high-throughput molecular biology manipulations (e.g., cloning/sequencing) for the generation (and subsequent interrogation) of large libraries coding for distinct peptide sequences within the context of a given MHC allele. This platform leverages the “universal” nature of the single chain construction. In this paradigm, the only difference amongst the library is the 27-nucleotide sequence encoding the 9-mer-peptide itself (e.g., the epitope). This unique sequence can be amplified with “universal primers” (labeled as Forward/Reverse in FIG. 2) and readily identified by deep sequencing and subsequent translation following T-cell challenge [31-34]. Notably, the nucleotide sequence forming the consensus region for “universal” primer annealing has been modified away from the native B2M nucleotide sequence to avoid background amplification from the endogenous B2M pool (i.e. B2M from the HEK cell).

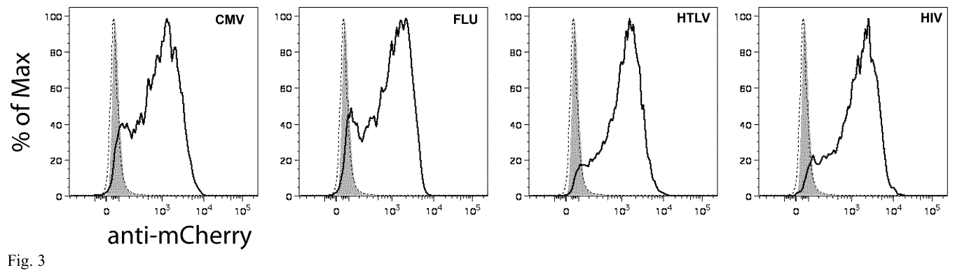

MHC controls. Initial feasibility studies within our group leverage 5 known pathogenic HLA-A02 restricted epitopes linked to 5 independent viral pathogens (cytomegalovirus pp65 protein residues 495-504 [henceforth referred to as CMV], Influenza matrix protein 58-66 [FLU], Epstein-Barr virus BMLF1 259-267 [EBV], Human T-lymphotropic virus Tax 11-19 [HTLV] and HIV gag p17 76-84 [HIV]). HEK293 stable cell lines were generated by lentiviral transduction of virus carrying sc-pMHC constructs bearing a surface mCherry expression proxy anchored to the membrane through the native human class-I heavy chain transmembrane domain as illustrated in FIG. 2. Surface expression of our constructs was validated through fluorescence-activated cell sorting (FACS) analysis monitoring mCherry proxy expression and anti-mCherry surface expression (4 of which are illustrated in FIG. 3), and supports that the constructs express and are properly targeted to the plasma membrane. Notably, the EBV control peptide was added subsequent to the generation of this figure, however the surface expression profile of EBV mirrors those observed for the other 4 controls (data not shown).

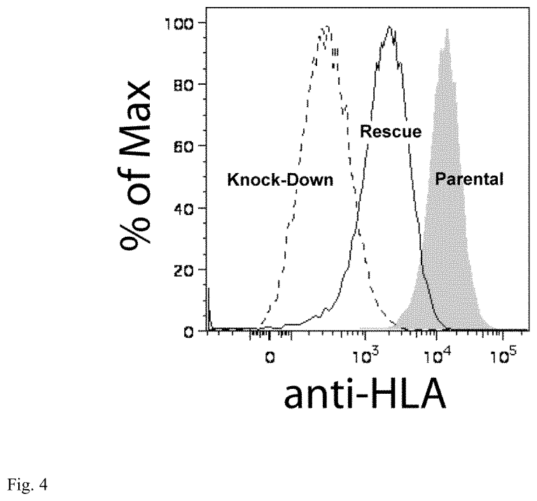

In this system, surface presentation of mCherry is an indicator of proper folding of the MHC construct, as unfolded proteins are more often trapped/retained in the ER/Golgi [35], however a direct assessment on MHC folding is of course desirable. As HEK293 cells natively express HLA and B2M molecules, direct staining against surface B2M or HLA to monitor proper folding is challenging. To ensure that the single chain membrane anchored MHC design results in properly folded material shRNA hairpins targeting the 5′ untranslated region (UTR) of native human B2M (pGIPZ clone VA282 [catalogue number RHS4430-101098345], knock-down cells provided by the Einstein shRNA core facility) were leveraged to down regulate the endogenous expression of B2M and surface MHC within HEK cells (B2M was chosen for down regulation as it is essential for MHC folding/localization regardless of MHC isotype). This implementation does not contain the 5′ UTR to promote persistent expression of the integrated constructs. As shown in FIG. 4, greater than 95% of native HLA was effectively down-regulated using the shRNA strategy as monitored by surface staining against a conformationally dependent anti-Class I MHC antibody (the mAb W6/32 used requires that both MHC and B2M are properly folded for binding [36]. Notably proper processing of the leader peptide and epitope presentation are a requirement of MHC stability/folding). MHC surface expression was restored upon transduction with a representative construct from the library (CMV). Taken together, this suggests that the constructs are both properly localized (as monitored by anti-mCherry) and well folded (as monitored by anti-HLA, FIG. 4).

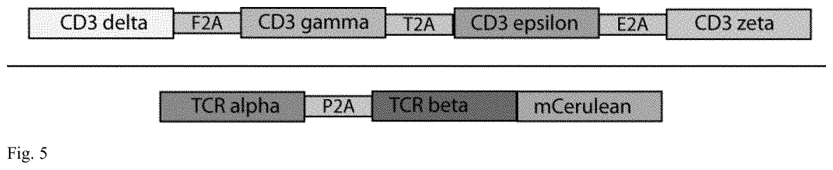

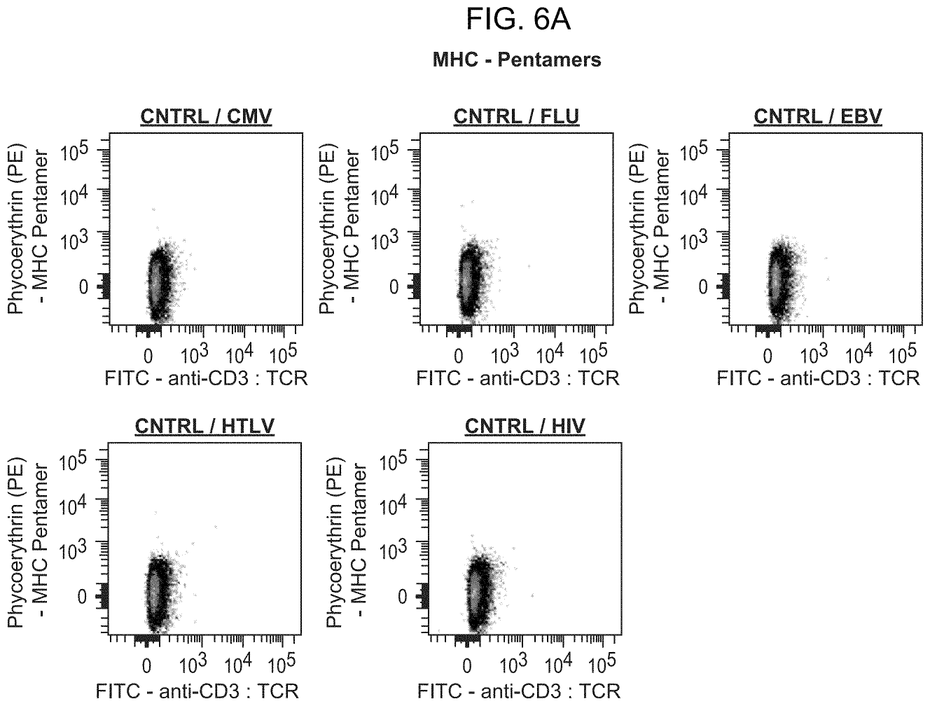

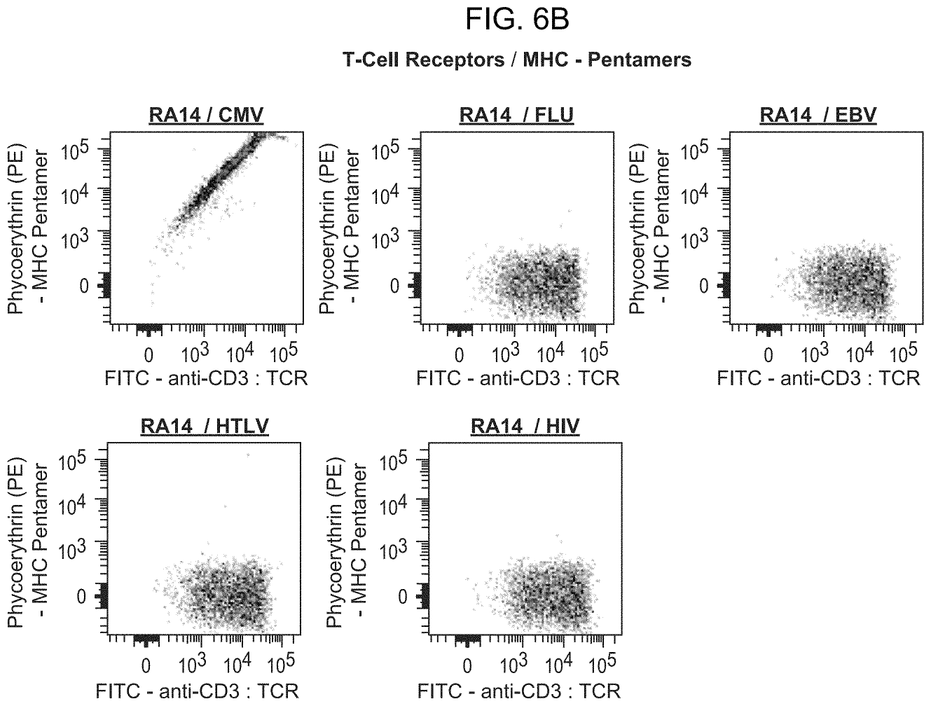

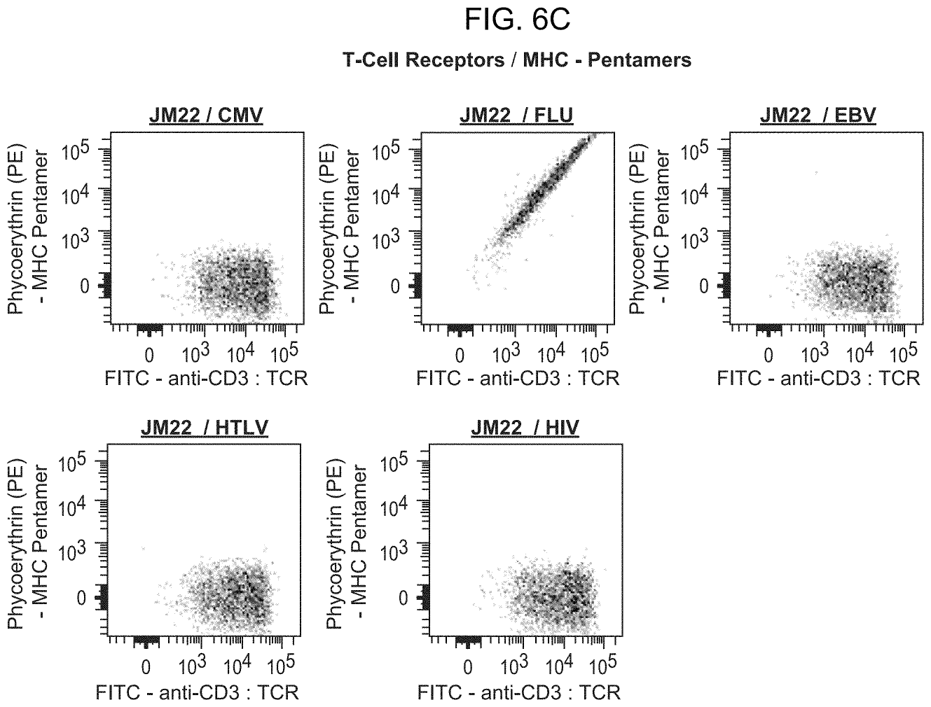

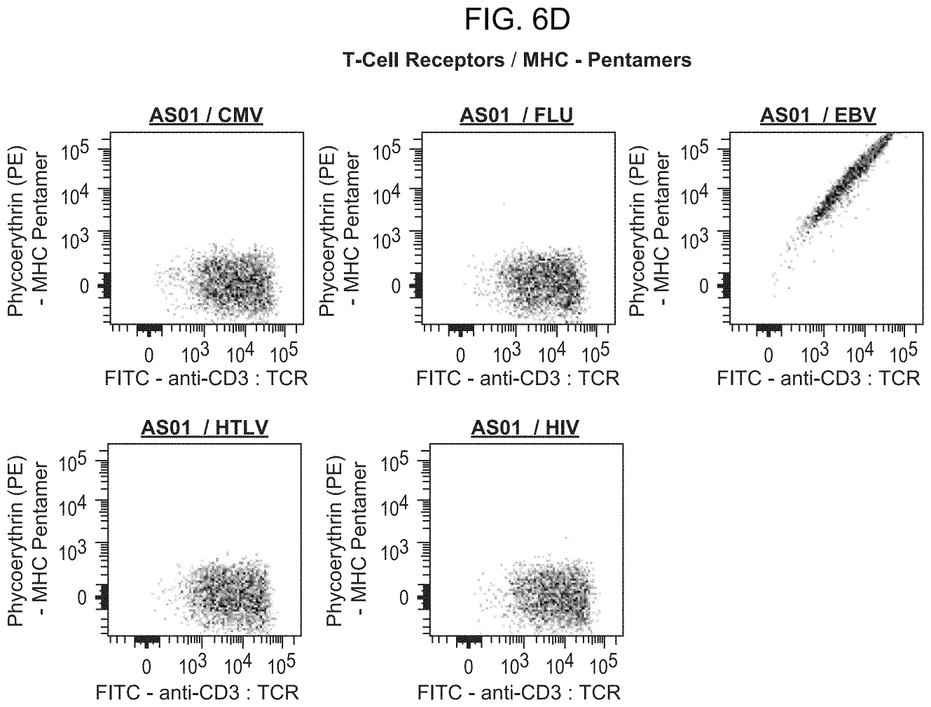

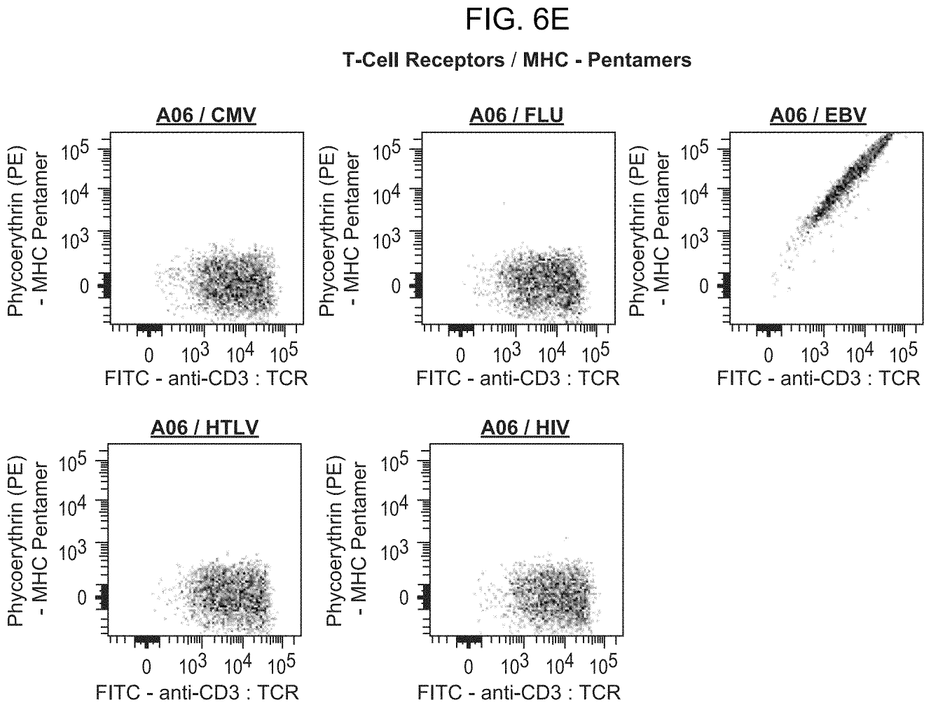

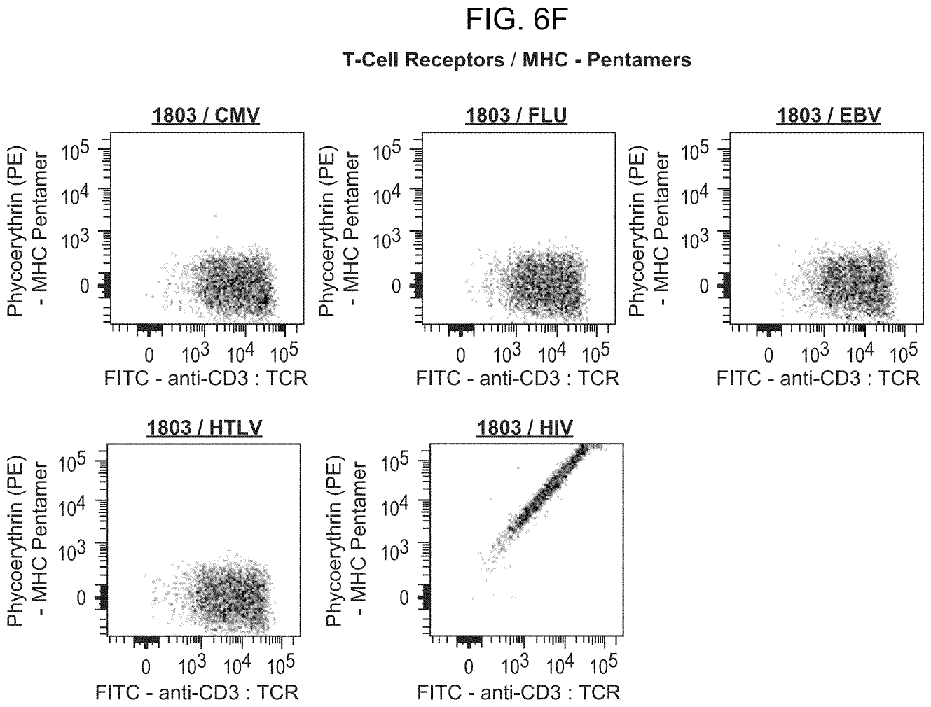

TCR controls. Given the extensive use of suspension adapted HEK cell lines within this lab, HEK cells were naturally chosen as the expression host for generation of control TCR lines. HEK 293 cells do not endogenously express TCR genes, nor are they capable of expressing TCR constructs without modification (as observed by ourselves [data not shown] and others [37]). The TCR is a disulfide-linked membrane-anchored heterodimer (alpha/beta chains) expressed as part of a complex with the invariant CD3 chain molecules. The CD3 chains, together with the TCR, form what is known as the T-cell receptor complex. The full complex is required for proper expression and plasma membrane localization. To allow for expression of control TCRs, lentiviral co-transduction techniques were utilized, wherein one lentiviral construct harbors the full CD3 gene cassette linked by various viral 2A “self-cleaving” peptides [37] (FIG. 5, top) and the second carries the TCR alpha and beta chains linked by a single viral P2A peptide to allow for stoichiometric expression as the P2A peptide shows the highest “cleavage” efficiency in mammalian cells [2]. The mCerulean (BLUE) expression proxy follows the beta chain transmembrane segment (FIG. 5, bottom). Proof-of-principle studies employ the 5 cognate TCRs for the HLA molecules discussed above (TCR RA14 [binds to CMV peptide], JM22 [FLU], AS01 [EBV], A06 [HTLV] and 1803 [HIV]). Surface expression of the constructs was confirmed by anti-TCR as well as anti-CD3 antibody staining (data not shown) and active T-cell complex formation confirmed through cognate MHC pentamer staining (FIG. 6, pentamers purchased from ProImmune). Untransduced cells were used as a negative control.

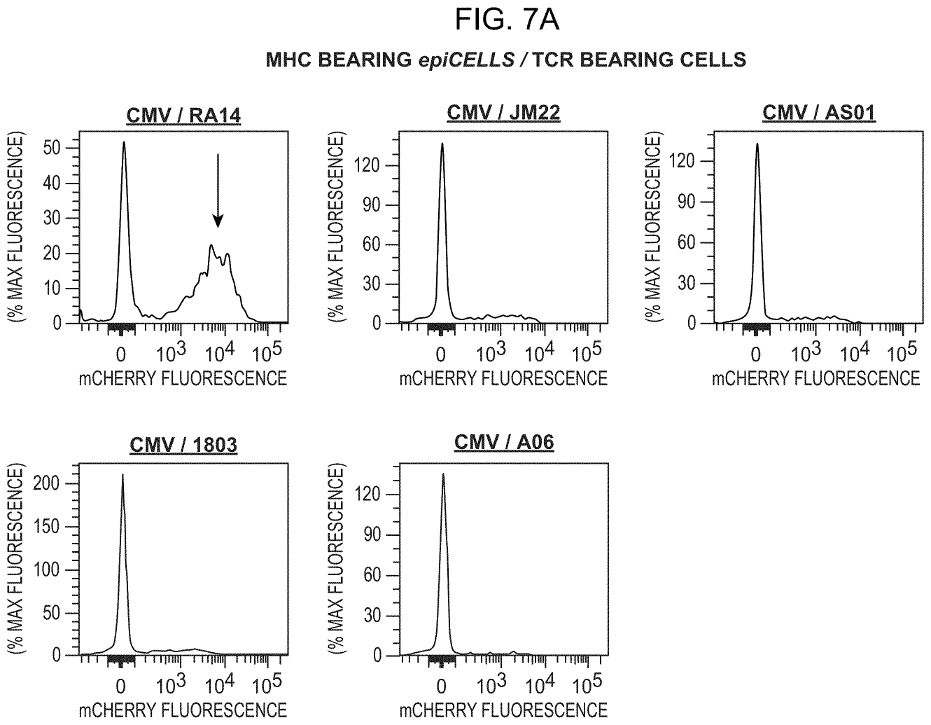

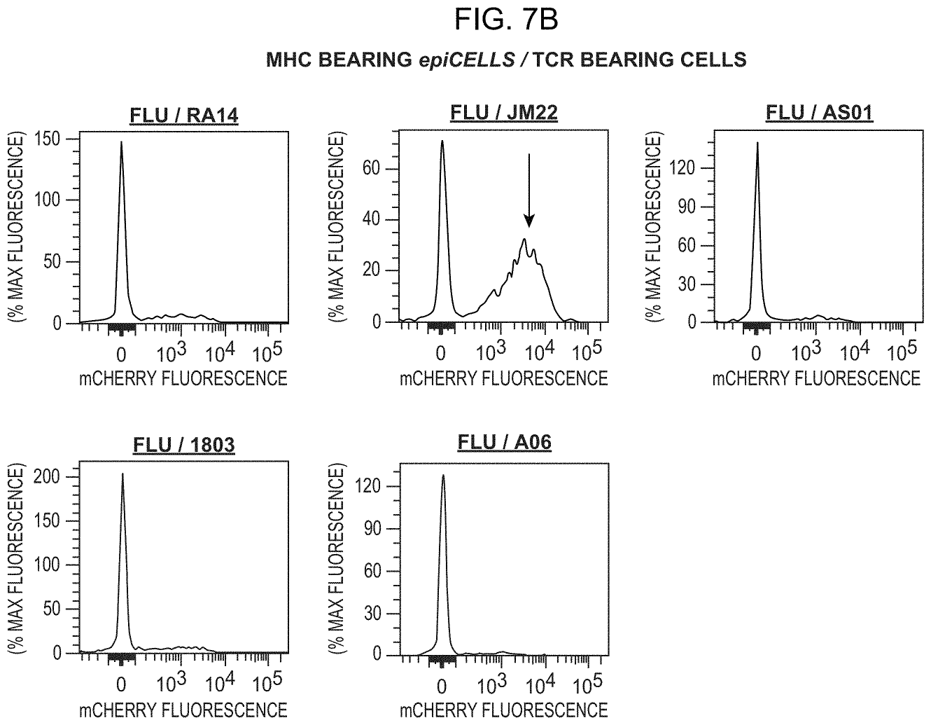

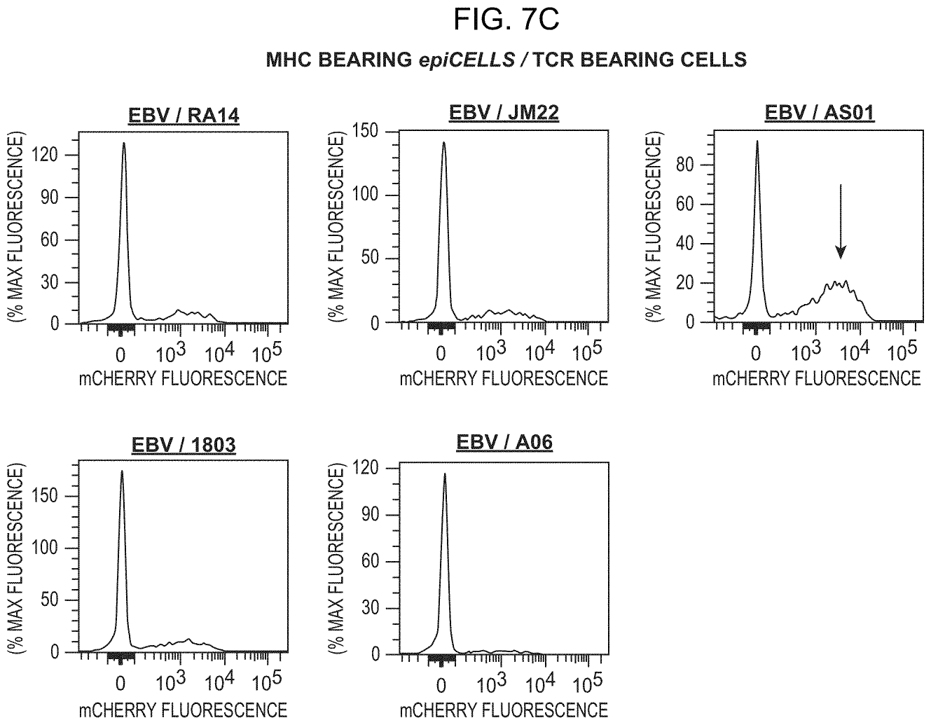

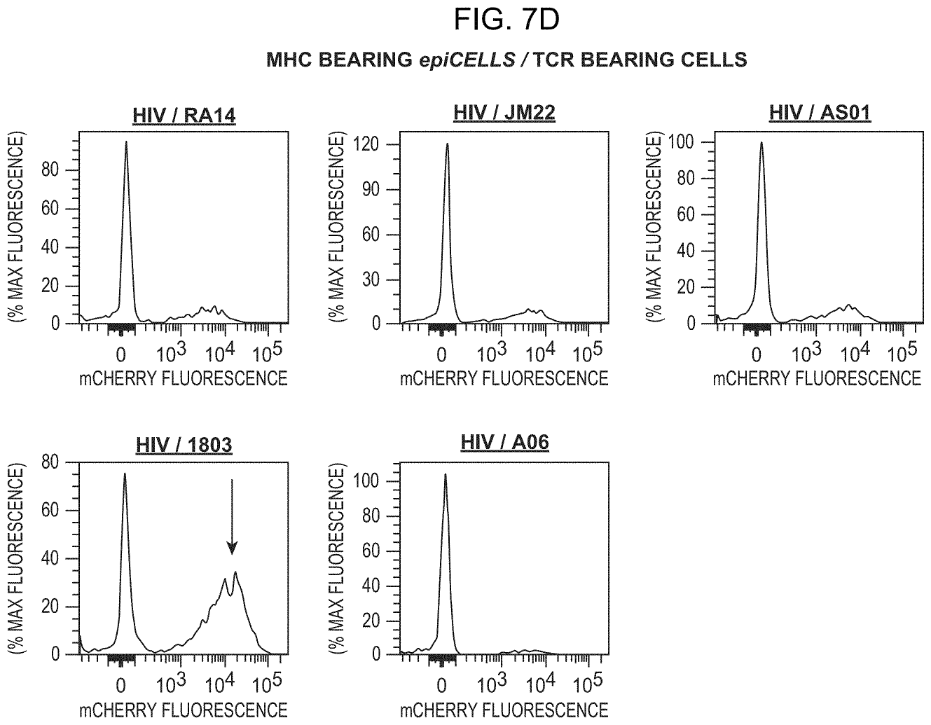

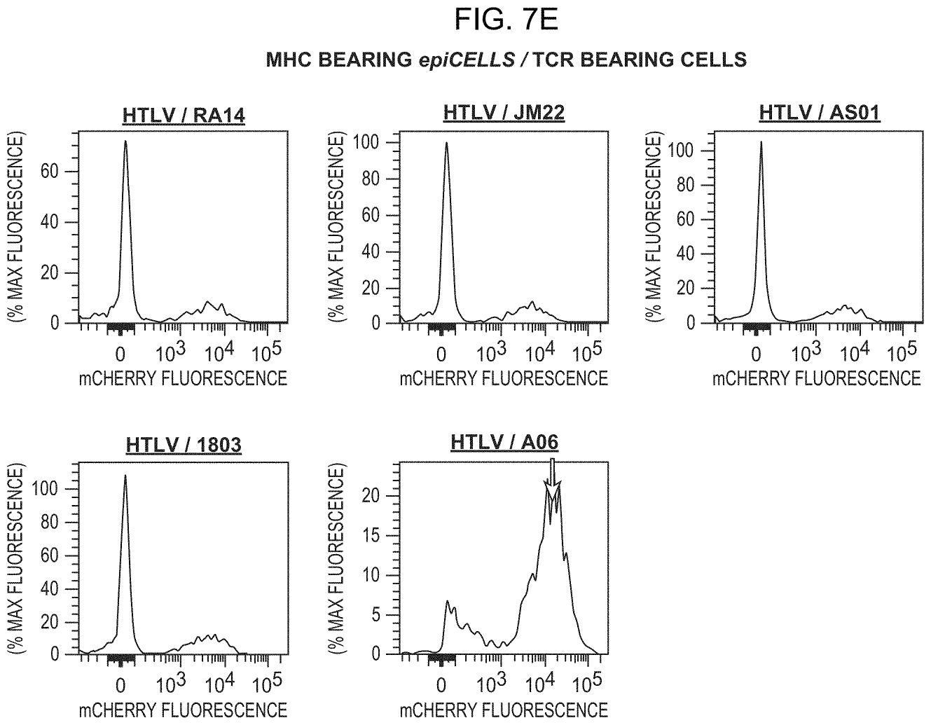

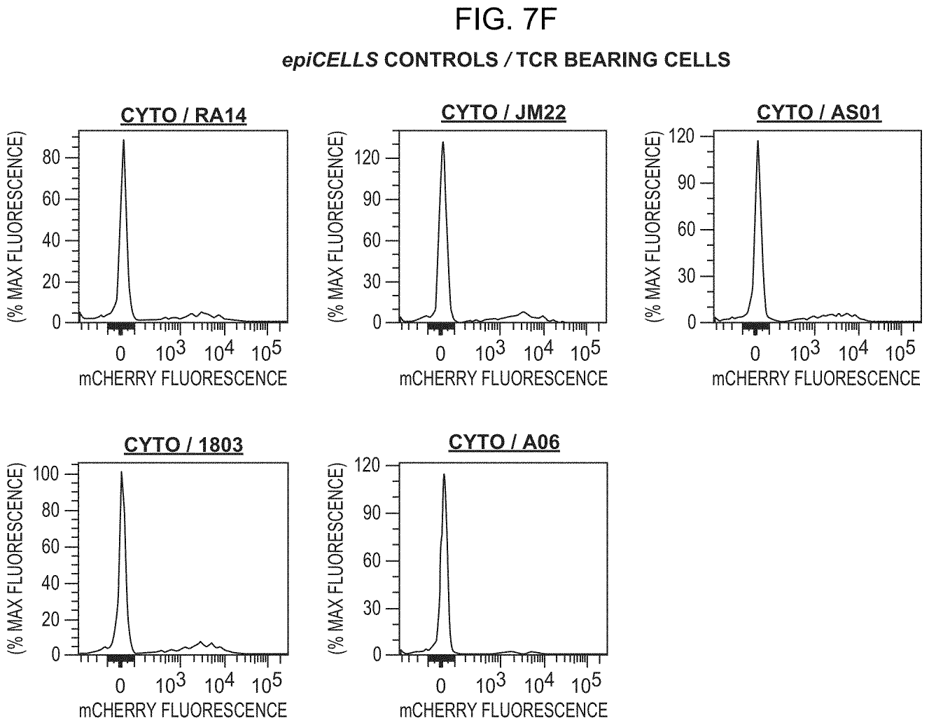

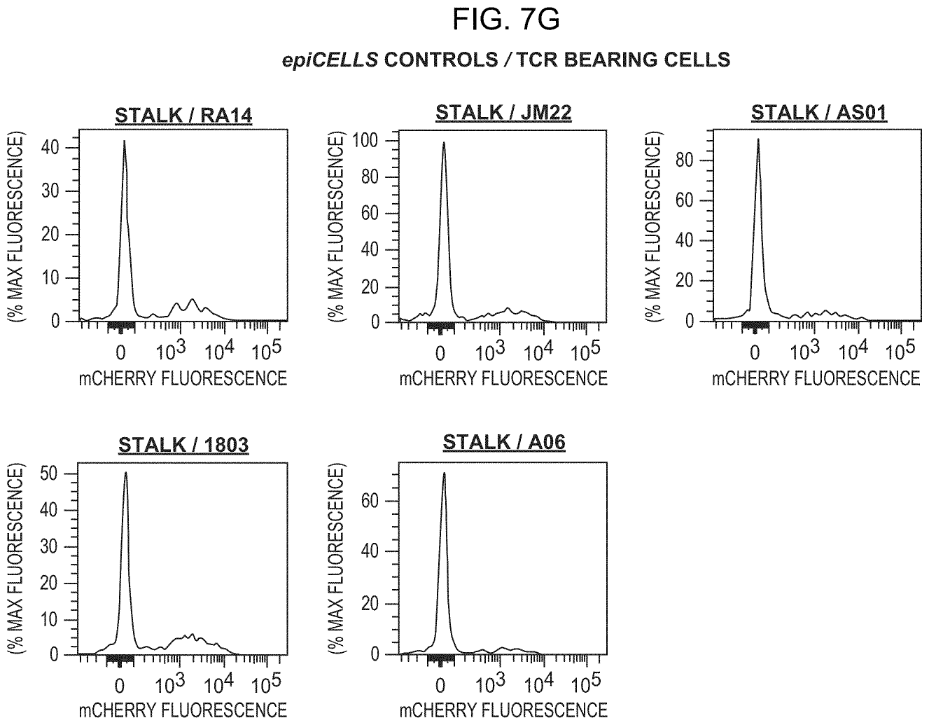

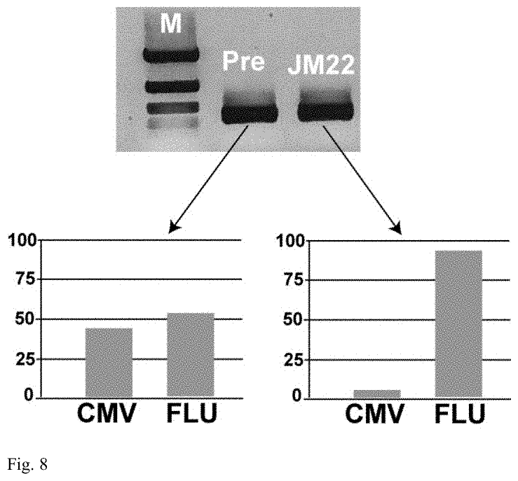

The epiCELL screening platform. While traditional MHC tetramer- (or the more recent pentamer-) based presentation affords enhanced avidity relative to single proteins, the expression of the query protein on the plasma membrane of eukaryotic cells is expected to provide greater antigen density, significantly higher avidity and expanded dynamic range for detecting weaker pMHC:TCR interactions. The 5 TCRs were individually expressed to complement the 5 cognate sc-pMHC epiCELLS. Cytoplasmic mCherry (labeled as CYTO) and surface expressed mCherry (without the MHC, labeled as STALK) were used as negative controls. Flow cytometric analysis of the individual and mixed populations clearly demonstrated a significant increase (as much as 100-fold, A06:HTLV interaction) in signal representing specific cell-cell interactions only when cells expressing cognate MHC:TCR pairs were both present (FIG. 7). Next two of the epiCELLs (CMV and FLU) were pooled, challenged with independent TCR bearing HEK cells (JM22 only), and sorted on the conjugates formed. The genomic DNA from each pool was extracted and subjected to ˜30 cycles of PCR using universal primers targeting flanking regions around the epitope (FIG. 2, above). The resulting PCR bands are shown in FIG. 8 (top), these amplicons were submitted for library preparation (e.g., addition of multiplexed TruSeq indexed adapters) and subsequent next generation sequencing (NGS) was performed (illumina MiSeq). Library preparation and sequencing was performed at the Einstein Epigenomics core facility. The resulting FASTQ files from the NGS run were analyzed and epitopes readily identified (FIG. 8, bottom). For each, the absolute number of epitope sequences observed were counted and normalized as a percent of ALL observed NGS reads that pass our QC filter. Notably, CMV and FLU was selected for initial screening as these were the first validated constructs in the library.

In a further example, an exhaustive screen against all overlapping 9-mers representing the EBV BMLF1 protein (a pool of ˜400 epiCELLS) can be surveyed to identify immunodominant signatures from EBV infected patients (this target was chosen as ˜95% of adults >35 years old maintain EBV reactive peripheral T-cells).

The epiCELL platform cab be used in defining the entire ensemble of biologically relevant T-cell epitopes associated with human disease. Next-generation-sequencing (NGS)-based epiCELL platform for epitope mapping can be combined with larger “combinatorial” libraries to allow for extension to mimotope screening against select TCRs to identify binders with altered affinities/kinetics, and further extended to the survey of all immunologic reactivities within a single patient sample simultaneously and with a sensitivity approaching comprehensive coverage. The identification of peptide antigen epitopes (and mimotopes) is an important first step in identifying, isolating and modulating class I MHC restricted T-cells involved in protective and pathological immune responses. Notably, the methods described can easily be extended/modified to an analogous exhaustive survey of Class-II (CD4+ T-cell) reactivities.

### Example 2

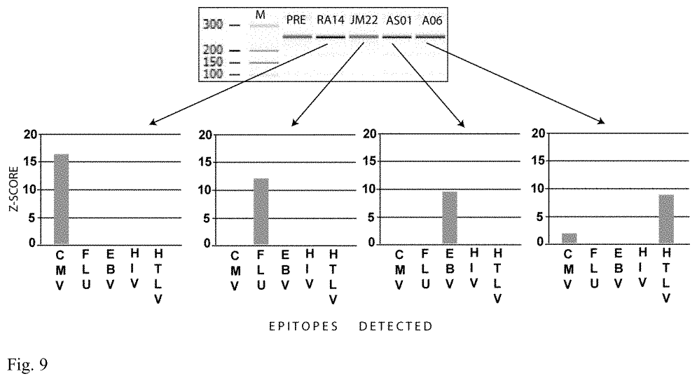

In this experiment, lentiviruses for all 5 MHC bearing epiCELLS (CMV, FLU, EBV, HIV, HTLV) were transduced separately, cells pooled in equal ratios and challenged against 4 cognate TCR bearing cells (RA14, JM22, ASO1, A06) independently and sorted on the conjugates formed. The genomic DNA from each pool was extracted and subjected to ˜30 cycles of PCR using universal primers targeting flanking regions around the epitope. The resulting PCR bands are shown in FIG. 9 (top), these amplicons were submitted for library preparation (e.g., addition of multiplexed TruSeq® indexed adapters) and subsequent next generation sequencing (NGS) was performed (Illumina MiSeq). Library preparation and sequencing was performed at the Albert Einstein College of Medicine epigenomics core facility. The resulting FASTQ files from the NGS run were analyzed and epitopes readily identified (FIG. 9, bottom). Epitopes identified within the pre-sorted population (the library) was within a range from 16-23% (data not shown). For each of the TCR challenged data sets, the absolute number of epitope sequences observed were counted and normalized as a percent of all observed NGS reads that pass the QC filter and was used to calculate a Z-score using the mean and standard deviation parameters taken from the pre-sorted pool. These results highlight the utility of the epiCELL platform and show the robustness of the screen even using un-optimized binding/washing protocols.

### Example 3

The utility of epiCELL can be extended to include not only single chain constructs (with and without bivalent presentation through Fc fusion), but also split constructs (synTacs) to allow for local presentation of multiple protein or peptide fragments within the context of epiCELLs. Furthermore, the use of small plasma membrane containing fragments derived from epiCELL pools (e.g., microvessicles, exosomes, viral like particles [VLPs] and retroviruses [e.g., lentivirus, etc.]) have allowed for vast decreases in reaction volumes for screening (these viral particle-based approaches are sometimes referred to herein as “viratopes”). These expression pools (epiCELLS or viratopes) are challenged with T-cells from healthy, infected, cured and immunized patients to identify those epitopes that are directly relevant to disease, diagnosis, treatment, neutralization and monitoring of disease progression and therapeutic response.

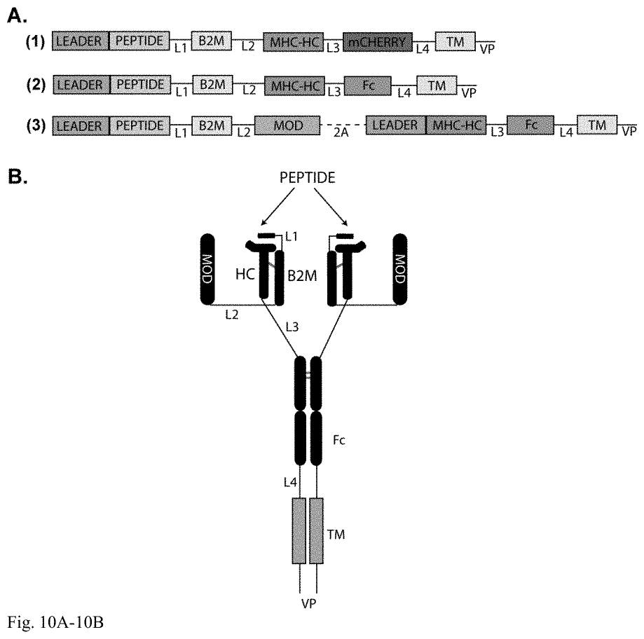

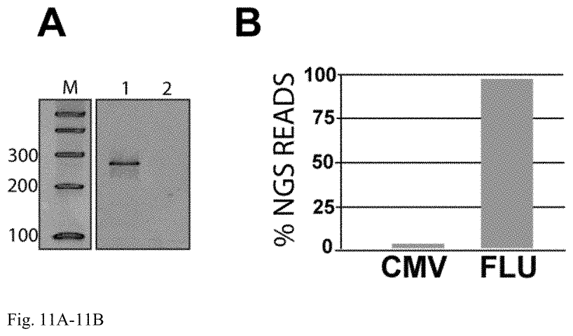

Exemplary variants continue to utilize a sequence the same as a native human B2M leader sequence immediately followed by a candidate peptide epitope that is covalently linked to the B2M molecule through linker L1 (illustrated in FIG. 10A). However, the 3 new constructs differ in their overall architecture and covalent organization. A first variant (See FIG. 10A.(1)) is analogous to traditional epiCELL based presentation, but with the addition of a viral packaging signal at the extreme C-terminal end (e.g, GP41 envelope protein residues 706-713, etc., labeled as VP). The presence of the VP sequence has no consequence in the context of traditional epiCELL based screening, but does allow for packaging into viral like particles (VLPs) or retroviruses (e.g., lentivirus) when budded from epiCELL pools. To increase local valency of surface expressed constructs, an Fc-Fusion-based construction is used (FIG. 10A. (2), e.g., human IgG1 Fc, murine IgG2a Fc, etc.), again terminating in a VP sequence to allow for viral packaging. As the proxy for surface expression (i.e., mCherry) has been removed from this variant, epitopes for traditional antibodies (e.g., FLAG, MYC, etc.) have been placed in linker L4 to allow for detection of plasma membrane localization via antibody staining. These currently described constructs (A.1 and A.2) have utilized a single chain construction and as such are limited with respect to the ability to extend the system through alternative protein linkages for screening purposes. To increase the modularity/flexibility of the epiCELL screening platform through the inclusion of additional protein or peptide linkages, a synTac-based expression construct, also developed in this laboratory, is utilized. Briefly, the strategy underlying synTac splits the MHC construct into respective heavy and light chains, with fusion of both peptides and proteins to various termini (FIG. 10A. (3)) and schematically represented FIG. 10B). This construction results in covalent fusion of the peptide epitope to the N-terminus of the light chain (B2M) followed by a carboxy terminal extension of the light chain to our MOD effector molecule, FIG. 10B. In this scenario the heavy chain (HLA-molecule) is fused to the Fc region. All components associate during production within eukaryotic cells (e.g., HEK, CHO) and self-assemble. Notably, the two chains are covalently tethered through disulfide bonds (shown as RED lines). The MOD in this case can be any protein, peptide or other chemical entity required for screening. Examples are antibody epitopes (FLAG, MYC etc.) for secondary staining, fluorescent proteins (GFP, mFruit, etc.) for direct fluorescent detection, nucleic acid binding proteins or comodulatory proteins. All constructs are localized to the plasma membrane through a native Class-I Heavy Chain transmembrane domain (TM, although this could be any human, murine or other TM domain). Universal primers are used to amplify the unique 27-nucleotide sequence (9-mer peptide) found in a current construct following T-cell challenge to directly identify disease relevant epitopes. However, peptides of lengths varying from, but not restricted to, 5-20 amino acids are candidate epitopes.

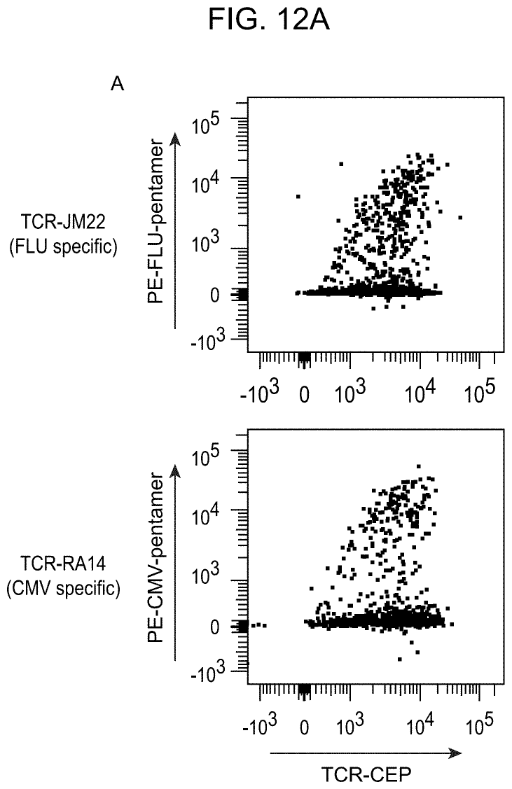

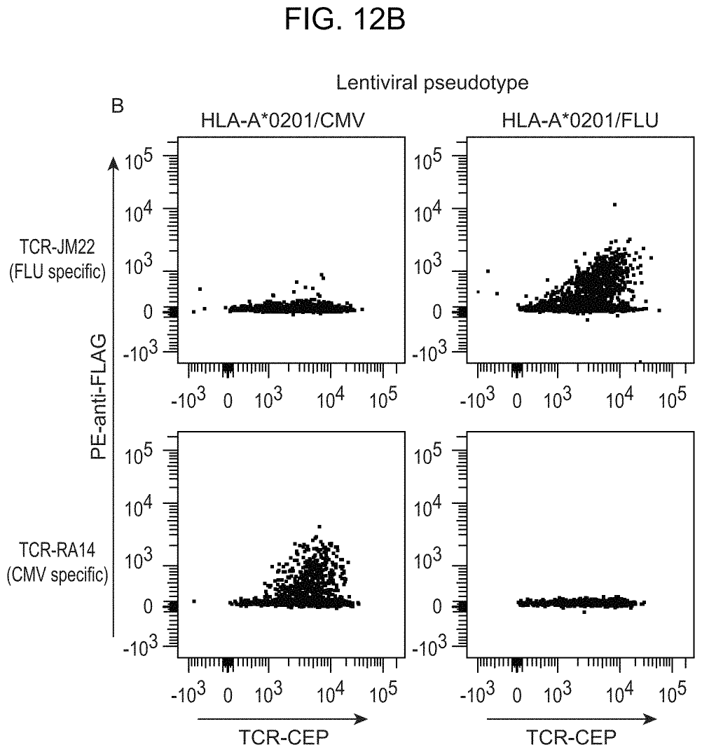

Extending the epiCELL screening platform for lentiviral display: Viratope. In the context of full combinatorial screening (e.g., when the peptide sequence is fully randomized), volumes ensuring full library coverage (i.e., 10⁹ epitopes) can range from 10-100 milliliters when using traditional epiCELLs. This requirement results predominantly from the relatively large size of the HEK cells used for epiCELL display, coupled with poor cellular viability at high concentrations (e.g., at concentrations greater than 10 million per ml). Retroviruses (e.g., lentivirus) are routinely generated from HEK cells through transfection with a packaging plasmid that contains specific virus-encoded genes (termed helper plasmid), along with an envelope protein (VSV-G, etc.). Notably, retroviral particles budded from these cells are stable at extreme concentrations (greater than 1 billion per mL). To take advantage of decreased reaction volumes and enhanced stability, we leveraged the viral packaging signals (VP) within our surface displayed MHC constructs to allow for packing into viral particles following transfection of epiCELL pools with helper plasmid alone (no addition of envelope plasmid), effectively pseudotyping the budded lentivirus with peptide MHC. Specifically, single chain constructs (FIG. 10A. (2)) composed of a peptide epitope linked to beta-2 microglobulin (B2M), HLA-A*0201, and human IgG1 Fc were substituted for the envelope component of a third generation lentiviral transfection system. The constructs also contained a FLAG epitope tag for detection by secondary antibodies (placed in the L4 linker region). The peptide epitopes presented in the context of HLA-A*0201 were either the NLVPMVATV (SEQ ID NO:9) peptide epitope from human cytomegalovirus (CMV) or the GILGFVFTL (SEQ ID NO:10) peptide epitope from influenza (FLU). Harvested lentivirus were concentrated 100× by ultracentrifugation and stored at 4 degrees Celsius. The genomic RNA (as opposed to DNA) from each viratope pool was extracted and subjected to one round of reverse transcription (RT) followed by ˜30 cycles of PCR using universal primers targeting flanking regions around the epitope. The resulting PCR bands are shown in FIG. 11A. Notably, a PCR band is only observed in the presence of an initial RT step (lane 1) and is absent when reverse transcriptase is omitted (lane 2), demonstrating the generation of competent (e.g., RNA-loaded) retrovirus. These amplicons were submitted for library preparation (e.g., addition of multiplexed TruSeq indexed adapters) and subsequent next generation sequencing (NGS) was performed (illumina MiSeq) to ensure compatibility of the process with epiCELL screening. Library preparation and sequencing was performed at the Einstein Epigenomics core facility. The resulting FASTQ files from the NGS run were analyzed and epitopes readily identified (FIG. 11B). Analogous to previous epiCELL binding experiments, viratope was then applied to HEK cells previously transfected with either a specific or irrelevant T cell receptor (TCR). Excess viratope particles were washed from cells and the remaining cell-bound lentivirus was detected via a PE-conjugated anti-FLAG antibody. Viratope pseudotyped with the cognate, but not the irrelevant epitope, bound to their respective TCR-expressing HEK cells in a manner comparable to staining by specific peptide-MHC pentamers (FIG. 12), demonstrating the specificity and general utility of viratope particles derived from epiCELL pools for epitope screening.

The current next-generation-sequencing (NGS) based epiCELL/viratope platform for epitope mapping can be combined with larger “combinatorial” libraries to allow for extension to mimotope screening against select TCRs to identify binders with altered affinities/kinetics, and further extended to the survey of ALL immunologic reactivities within a single patient sample simultaneously and with a sensitivity approaching comprehensive coverage. The identification of peptide antigen epitopes (and mimotopes) is important in identifying, isolating and modulating class I MHC restricted T-cells involved in protective and pathological immune responses. The methods described above are readily extended to an analogous survey of Class-II (CD4⁺ T-cell) reactivities.

### REFERENCES

1. Robins H S, S. S., Campregher P V, Turtle C J, Andriesen J, Riddell S R, Carlson C S, Warren E H, Overlap and effective size of the human CD8+ T cell receptor repertoire. Sci Transl Med, 2010. 2(47): p. 47ra64.
2. Jin Hee Kim, S.-R. L., Li-Hua Li, Hye-Jeong Park, Jeong-Hoh Park, Kwang Youl Lee, Myeong-Kyu Kim,Boo Ahn Shin,Seok-Yong Choi, High Cleavage Efficiency of a 2A Peptide Derived from Porcine Teschovirus-1 in Human Cell Lines, Zebrafish and Mice. PlosOne, 2011. 6(4): p. e18556.
3. Wen F, Z. H., Construction and screening of an antigen-derived peptide library displayed on yeast cell surface for CD4+ T cell epitope identification. Methods Mol Biol, 2013. 1061: p. 245-64.
4. Yibing Wang, A. R., Ryan Heiser, Janice White, Frances Crawford, Philippa Marrack, and John W. Kappler, Using a baculovirus display library to identify MHC class I mimotopes. PNAS, 2004. 102(7): p. 2476-2481.
5. S J, R., Peptide libraries for T cell epitope screening and characterization. J Immunol Methods, 2002. 267(1): p. 71-7.
6. Tim BeiBbarth, J. A. T.-D., Gordon K. Smyth, Terence P. Speed, and Robert P. Anderson, A systematic approach for comprehensive T-cell epitope discovery using peptide libraries. Bioinformatics, 2005. 21: p. i29-i37.
7. Verena Rubio-Godoy, V.r.D., Yingdong Zhao, Richard Simon, Philippe Guillaume, Richard Houghten, Pedro Romero, Jean-Charles Cerottini, Clemencia Pinilla, and Danila Valmori, Positional Scanning-Synthetic Peptide Library-Based Analysis of Self- and Pathogen-Derived Peptide Cross-Reactivity with Tumor- Reactive Melan-A-Specific CTL. J Immunol, 2002. 169: p. 5696-5707.
8. Gokhan Deviren, K. G., Michael E. Paulaitis and Jonathan P. Schneck, Detection of antigen-specific T cells on p/MHC microarrays. J. Mol. Recognit, 2007. 20: p. 32-38.
9. Cox, A., Skipper, J, Chen, Y, Henderson, R A, Darrow, T L, Shabanowitz, J, Engelhard, V H, Hunt, D F, Slingluff, C L Jr, Identification of a peptide recognized by five melanomaspecific human cytotoxic T cell lines. Science, 1994. 264: p. 716-719.
10. Gerald T. Nepom, J. D. L., Forest M. White Susan Masewicz, Jarrod A. Marto, Andrew Herman, C. John Luckey, Ben Falk, Jeffrey Shabanowitz, Donald F. Hunt, Victor H. Engelhard, and Barbara S. Nepom, Identification and modulation of a naturally processed T cell epitope from the diabetes-associated autoantigen human glutamic acid decarboxylase 65 (hGAD65). PNAS, 2001. 98(4): p. 1763-1768.
11. Stern, L., Characterizing MHC-associated peptides by mass spectrometry. J Immunol., 2002. 2007(179): p. 2667-2668.
12. Evan W Newell, N. S., Nitya Nair, Brian A Kidd, Harry B Greenberg, Mark M Davis, Combinatorial tetramer staining and mass cytometry analysis facilitate T-cell epitope mapping and characterization. Nature Biotechnology, 2013. 31: p. 623-629.
13. Katherine S. Garmin, J. R. N.a.A. P., Genomic strategies for personalized cancer therapy. Hum. Mol. Genet., 2007. 16(R2): p. R226-R232.
14. Andrew H Sims, K. R. O., Robert B Clarke, and Anthony Howell, High-throughput genomic technology in research and clinical management of breast cancer. Exploiting the potential of gene expression profiling: is it ready for the clinic? Breast Cancer Research, 2006. 8(5): p. 214-221.
15. Muhammed Murtaza, S.-J. D., Dana W. Y. Tsui, Davina Gale, Tim Forshew, Anna M. Piskorz, Christine Parkinson, Suet-Feung Chin, Zoya Kingsbury, Alvin S. C. Wong, Francesco Marass, Sean Humphray, James Hadfield, David Bentley, Tan Min Chin, James D. Brenton, Carlos Caldas & Nitzan Rosenfeld, Non-invasive analysis of acquired resistance to cancer therapy by sequencing of plasma DNA. Nature, 2013. 497: p. 108-112.
16. Espina V, D. K., Cowherd S, Petricoin E F 3rd, Liotta L A, Use of proteomic analysis to monitor responses to biological therapies. Expert Opin Biol Ther., 2004. 4(1): p. 83-93.
17. Moulay A. Alaoui-Jamali, D. P., Proteomic Profiling of Tyrosine Kinases as Pharmacological Endpoints for Targeted Cancer Therapy. Cancer Proteomics, ed. S. S. Daoud. 2008: Humana Press.
18. Daniel K. Nomura, M. M. D. B. F. C., Activity-based protein profiling for biochemical pathway discovery in cancer. Nature Reviews Cancer, 2010. 10: p. 630-638.
19. Donal J. Brennan, D. P. O. C., Elton Rexhepaj, Fredrik Ponten & William M. Gallagher, Antibody-based proteomics: fast-tracking molecular diagnostics in oncology. Nature Reviews Cancer, 2010. 10: p. 605-617.
20. T D., R., The current status and future potential of personalized diagnostics: Streamlining a customized process. Biotechnol Annu Rev, 2008. 14: p. 411-422.
21. Robins, H., Immunosequencing: applications of immune repertoire deep sequencing. Current Opinion in Immunology, 2013. 25: p. 1-7.
22. F A., W., TNF inhibition as therapy for rheumatoid arthritis. Expert Opin Investig Drugs, 2002. 11(7): p. 947-953.
23. Mansh, M., Ipilimumab and cancer immunotherapy: a new hope for advanced stage melanoma. Yale J Biol Med, 2011. 84(4): p. 381-389.
24. Larsen C P, P. T., Adams A B, Tso P, Shirasugi N, Strobert E, Anderson D, Cowan S, Price K, Naemura J, Emswiler J, Greene J, Turk L A, Bajorath J, Townsend R, Hagerty D, Linsley P S, Peach R J., Rational development of LEA29Y (belatacept), a high-affinity variant of CTLA4-Ig with potent immunosuppressive properties. Am J Transplant, 2005. 5(3): p. 443-453.
25. Bluestone, J. A., E. W. St Clair, and L. A. Turka, CTLA4Ig: bridging the basic immunology with clinical application. Immunity, 2006. 24(3): p. 233-238.
26. Kevin Horgan, S. S., Monica Boirivant, Immunomagnetic Purification of T-Cell Subpopulations. Current Protocols in Immunology, 2009. 85: p. 7.4.1-7.4.9.
27. Karlsson A C, M. J., Younger S R, Bredt B M, Epling L, Ronquillo R, Varma A, Deeks S G, McCune J M, Nixon D F, Sinclair E., Comparison of the ELISPOT and cytokine flow cytometry assays for the enumeration of antigen-specific T cells. J Immunol Methods, 2003. 283(1-2): p. 141-153.
28. Yik Y. L. Yu, N. N., Lonnie Lybarger, Janet M. Connolly and Ted H. Hansen, Single-Chain Trimers of MHC Class I Molecules Form Stable Structures That Potently Stimulate Antigen-Specific T Cells and B Cells. The Journal of Immunology, 2002. 168(7): p. 3145-4149.
29. Sojung Kim, L. L., Curtis P. McMurtrey, William H. Hildebrand, Jon A. Weidanz, William E. Gillanders, Michael S. Diamond, and Ted H. Hansen, Single- Chain HLA A2 MHC Trimers That Incorporate an Immundominant Peptide Elicit Protective T Cell Immunity against Lethal West Nile Virus Infection. The Journal of Immunology, 2010. 184: p. 4423-4430.
30. Samanta D, M. G., Ramagopal U A, Chaparro R J, Nathenson S G, DiLorenzo T P, Almo S C., Structural and functional characterization of a single-chain peptide- MHC molecule that modulates both naive and activated CD8+ T cells. Proc Natl Acad Sci USA, 2011. 108(33): p. 13682-13687.
31. Whitehead, T. A., et al., Optimization of affinity, specificity and function of designed influenza inhibitors using deep sequencing. Nat Biotechnol, 2012. 30(6): p. 543-8.
32. Smith, A. M., et al., Competitive genomic screens of barcoded yeast libraries. J Vis Exp, 2011(54).
33. Lee, W., et al., Genome-wide requirements for resistance to functionally distinct DNA-damaging agents. PLoS Genet, 2005. 1(2): p. e24.
34. Pierce, S. E., et al., Genome-wide analysis of barcoded Saccharomyces cerevisiae gene-deletion mutants in pooled cultures. Nat Protoc, 2007. 2(11): p. 2958-74.
35. Satomi Nadanaka, H. Y., Fumi Kano, Masayuki Murata, and Kazutoshi Mori, Activation of Mammalian Unfolded Protein Response Is Compatible with the Quality Control System Operating in the Endoplasmic Reticulum. Molecular Biology of the Cell, 2004. 15: p. 2537-2548.
36. David N. Garboczi, D. T. H., and Don C. Wiley, HLA-A2-peptide complexes: Refolding and crystaliization of molecules expressed in Eschericia Coli and complexed with single antigenic peptides. Proc Natl Acad Sci USA, 1992. 89: p. 3429-3433.
37. Andrea L Szymczak, C. J. W., Yao Wang, Kate M Vignali, Smaroula Dilioglou, Elio F Vanin and Dario A A Vignali, Correction of multi-gene deficiency in vivo using a single ‘self-cleaving’ 2A peptide-based retroviral vector. Nature Biotechnology, 2004. 22: p. 589-594.

1.-82. (canceled)

83. A lentivirus or lentivirus-like particle comprising a heterologous polypeptide comprising, in order from N-terminus to C-terminus:
a) a peptide of from 8 to 12 amino acids in length, contiguous with;
b) a first linker peptide, contiguous with;
c) a β-2 microglobulin (β2M) polypeptide, contiguous with;
d) a second linker peptide, contiguous with;
e) a class I major histocompatibility complex (WIC) heavy chain, contiguous with;
f) a third linker peptide, contiguous with;
g) a fluorescent protein or an immunoglobulin Fc fragment, contiguous with;
h) a fourth linker peptide, contiguous with;
i) a mammalian transmembrane domain, contiguous with;
j) a viral packaging signal comprising the GP41 lentivirus envelope protein residues 706-713,
wherein the peptide of from 8 to 12 amino acids in length is displayed on the surface of the lentivirus or the lentivirus-like particle in complex with the β2M polypeptide and the class I MHC heavy chain.

84. The lentivirus or lentivirus-like particle of claim 83, wherein the class I MHC heavy chain is an HLA-A*0201 polypeptide.

85. The lentivirus or lentivirus-like particle of claim 83, wherein the first, second, third, and fourth linkers comprise the amino acid sequence GGGGS.

86. The lentivirus or lentivirus-like particle of claim 83, wherein the transmembrane domain is an class I MEW heavy chain transmembrane domain.

87. The lentivirus or lentivirus-like particle of claim 83, wherein the peptide of from 8 to 12 amino acids in length is NLVPMVATV or GILGFVFTL.

88. A plurality of the lentivirus or lentivirus-like particle of claim 83.

89. The plurality of claim 88, wherein the plurality comprises at least 2 different peptides of 8 to 12 amino acids in length.

90. The plurality of claim 88, wherein the plurality comprises at least 100 different peptides of 8 to 12 amino acids in length.

91. A method of identifying a T-cell epitope, the method comprising:
a) contacting a T-cell with the plurality of lentivirus or lentivirus-like particle of claim 88, under conditions that permit the T-cell to bind to one of the peptides having a length of from 5 to 20 amino acids, forming a complex between the T-cell and the virus or virus-like particle;
b) recovering the complex; and
c) sequencing the nucleic acid encoding the peptide having a length of from 5 to 20 amino acids present in the peptide-presenting cell present in the complex, thereby identifying the T-cell epitope.

92. The method of claim 91, wherein the T-cell is a peripheral T-cell obtained from a subject.

93. The method of claim 91, wherein the complex is recovered using a secondary antibody directed to an epitope in the lentivirus or lentivirus-like particle.

95. The method of claim 91, wherein the plurality of lentiviruses or lentivirus-like particles comprises at least 10³ different peptides having a length of from 5 to 20 amino acids.
